(19) United States
(12) Patent Application Publication
(10) Pub. No.: US 2022/0054748 A1
(43) Pub. Date: Feb. 24, 2022

(54) CONTROL MODEL FOR ARTIFICIAL PANCREAS

(71) Applicant: PRESIDENT AND FELLOWS OF HARVARD COLLEGE, Cambridge, MA (US)

(72) Inventors: Eyal DASSAU, Cambridge, MA (US); Dawei SHI, Cambridge, MA (US); Francis J. DOYLE III, Cambridge, MA (US)

(73) Assignee: PRESIDENT AND FELLOWS OF HARVARD COLLEGE, Cambridge, MA (US)

(21) Appl. No.: 17/283,859

(22) PCT Filed: Oct. 11, 2019
(86) PCT No.: PCT/US2019/055888

§ 371 Date: Apr. 8, 2021

Related U.S. Application Data
(60) Provisional application No. 62/745,880, filed on Oct. 15, 2018.

(51) Int. Cl. A61M 5/172

(52) U.S. Cl. CPC A61M 5/1723; A61M 2005/1726; A61M 2230/201; A61M 2205/52

(57) ABSTRACT

A multivariate parameter adaptation approach is disclosed for long-term use of an artificial pancreas using a dual-layer control scheme. The adaptation problem, which can be treated as an optimization problem with an unknown objective function and constraints, may be solved by the proposed BO-assisted multivariate optimization approach. Results showed that the algorithm was able to identify the improperly tuned parameters and smoothly adjust them for improved glucose regulation, despite lifestyle disturbances.

### CROSS REFERENCE TO RELATED APPLICATIONS

This application is a 371 National Phase Entry of International Patent Application No. PCT/US2019/055888, filed Oct. 11, 2019, which claims priority to U.S. Provisional Application No. 62/745,880, filed Oct. 15, 2018, titled MULTIVARIATE BAYESIAN OPTIMIZATION FRAMEWORK FOR LONG-TERM CONTROLLER ADAPTATION IN ARTIFICIAL PANCREAS, the contents of each of which are incorporated herein by reference in their entireties.

### STATEMENT REGARDING FEDERALLY SPONSORED RESEARCH

This invention was made with government support under NIH Grant Nos. UC4DK108483 and DP3DK104057 awarded by the U.S. Department of Health & Human Services. The government has certain rights in the invention.

### FIELD

The present invention is directed to control models for artificial pancreases, including both long term and short term adaptation of parameters.

### BACKGROUND

The following description includes information that may be useful in understanding the present invention. It is not an admission that any of the information provided herein is prior art or relevant to the presently claimed invention, or that any publication specifically or implicitly referenced is prior art.

Diabetes is a metabolic disorder that afflicts tens of millions of people throughout the world. Diabetes results from the inability of the body to properly utilize and metabolize carbohydrates, particularly glucose. Normally, the finely-tuned balance between glucose in the blood and glucose in bodily tissue cells is maintained by insulin, a hormone produced by the pancreas which controls, among other things, the transfer of glucose from blood into body tissue cells. Upsetting this balance causes many complications and pathologies including heart disease, coronary and peripheral artery sclerosis, peripheral neuropathies, retinal damage, cataracts, hypertension, coma, and death from hypoglycemic shock.

In patients with insulin-dependent diabetes, the symptoms of the disease can be controlled by administering additional insulin (or other agents that have similar effects) by injection or by external or implantable insulin pumps. The “correct” insulin dosage is a function of the level of glucose in the blood. Ideally, insulin administration should be continuously readjusted in response to changes in blood glucose level. In diabetes management, “insulin” instructs the body's cells to take in glucose from the blood. “Glucagon” acts opposite to insulin, and causes the liver to release glucose into the blood stream. The “basal rate” is the rate of continuous supply of insulin provided by an insulin delivery device (pump). The “bolus” is the specific amount of insulin that is given to raise blood concentration of the insulin to an effective level when needed (as opposed to continuous).

Presently, systems are available for continuously monitoring blood glucose levels by implanting a glucose sensitive probe into the patient. Such probes measure various properties of blood or other tissues, including optical absorption, electrochemical potential, and enzymatic products. The output of such sensors can be communicated to a hand held device that is used to calculate an appropriate dosage of insulin to be delivered into the blood stream in view of several factors, such as a patient's present glucose level, insulin usage rate, carbohydrates consumed or to be consumed, and exercise, among others. These calculations can then be used to control a pump that delivers the insulin, either at a controlled basal rate, or as a bolus. When provided as an integrated system, the continuous glucose monitor, controller, and pump work together to provide continuous glucose monitoring and insulin pump control.

Such systems at present require intervention by a patient to calculate and control the amount of insulin to be delivered. However, there may be periods when the patient is not able to adjust insulin delivery. For example, when the patient is sleeping, he or she cannot intervene in the delivery of insulin, yet control of a patient's glucose level is still necessary. A system capable of integrating and automating the functions of glucose monitoring and controlled insulin delivery would be useful in assisting patients in maintaining their glucose levels, especially during periods of the day when they are unable to intervene. A closed-loop system, also called the “artificial pancreas (AP), consists of three components: a glucose monitoring device such as a continuous glucose monitor (“CGM”) that measures subcutaneous glucose concentration (“SC”); a titrating algorithm to compute the amount of analyte such as insulin and/or glucagon to be delivered; and one or more analyte pumps to deliver computed analyte doses subcutaneously.

In some known zone model predictive control (MPC) approaches to regulating glucose, the MPC penalizes the distance of predicted glucose states from a carefully designed safe zone based on clinical requirements. This helps avoid unnecessary control moves that reduce the risk of hypoglycemia. The zone MPC approach was originally developed based on an auto-regressive model with exogenous inputs, and was extended to consider a control-relevant state-space model and a diurnal periodic target zone. Specifically, an asymmetric cost function was utilized in the zone MPC to facilitate independent design for hyperglycemia and hypoglycemia.

Throughout the development and adaptation of the MPC approaches, different controller adaptation methods have been utilized for AP design. Earlier studies considered basal rate and meal bolus adaptation by using run-to-run approaches based on sparse blood glucose (BG) measurements. The availability of CGM further provided the opportunity of designing adaptive AP utilizing advanced feedback controllers. For instance, a nonlinear adaptive MPC has been proposed to maintain normoglycemia during fasting conditions using Bayesian model parameter estimation. In other examples, a generalized predictive control (GPC) approach that adopted a recursively updated subject model has been employed on a bi-hormone AP; this approach has also been explored to eliminate the need of meal or exercise announcements.

A model predictive iterative learning control approach has also been proposed to adapt controller behavior with patient's day-to-day lifestyle. In some approaches, a multiple model probabilistic predictive controller was developed to achieve improved meal detection and prediction. A dynamic insulin-on-board approach has also been proposed to compensate for the effect of diurnal insulin sensitivity variation. A switched linear parameter-varying approach was developed to adjust controller modes for hypoglycemia, hyperglycemia and euglycemia situations. A run-to-run approach was developed to adapt the basal insulin delivery rate and carbohydrate-to-insulin ratio by considering intra- and inter-day insulin sensitivity variability.

A major drawback in the proposed AP designs is the difficulty in achieving satisfactory blood glucose regulation in terms of hyperglycemia and hypoglycemia prevention through designing smart control algorithms.

### SUMMARY

With the successful deployment of long-duration outpatient clinical trials [1]-[4], the AP technology has become mature enough to envision long-term home use applications. To improve usability in a home environment, an AP that can adapt with the growth, aging and lifestyle change of a patient needs to be developed.

Different approaches to AP adaptation have been investigated. Parameters adjusted usually include basal rate profiles that determine undisturbed glucose responses and carbohydrate (CHO) ratio profiles that determine meal responses. Automated basal rate modulation was considered in recent outpatient clinical trials [1], [2] and also in the first US Food and Drug Administration (FDA) approved commercial hybrid AP system in the US [3]. Pump setting adjustment with different degrees of automated adaptation and clinician effort was also considered in multiple clinical studies [5]-[7]. In a 12-week outpatient study [4], joint adaptation of basal rate and CHO ratio was performed based on an automated algorithm monitored by study physicians. An iterative learning approach was proposed in [8] to adapt the reference trajectory of a model predictive controller used for glucose regulation, which was tested in a pilot study [9]. In [10], [11], a run-to-run approach was proposed to update basal rate and meal bolus sizes based on sparse BG measurements; this approach was recently revisited in [12] to adapt basal rate at night and CHO ratio during the day based on CGM measurements, verified through multi-week simulations taking account of insulin sensitivity circadian rhythm. The problem of systematically optimizing AP adaptation to safely improve glucose control, however, remains to be explored.

Various challenges exist in designing AP adaptation algorithms. On one hand, glycemic metrics are influenced by disturbances caused by multiple sources in daily life (e.g., changing meal sizes, unannounced exercises and alcohol consumption). As a consequence, a subject with a poorly tuned AP may have satisfactory glucose metrics for some day when only small meals are consumed without physical exercises, while unsatisfactory metrics can be caused by a large unannounced meal even with a well-tuned AP. On the other hand, analytical relationships between tuning parameters and performance metrics are not known, which refuses the use of standard optimization methods in AP adaptation. Combined with the safety-critical nature of the problem and the urgent requirement on timely and efficient adaptation, this adds to the difficulty of algorithm design.

To overcome these challenges, the AP adaptation problem is studied utilizing a Bayesian optimization (BO) approach in this work. BO was developed to optimize unknown objective functions based on noisy measurements in the machine learning community [13], and has been recently used in on-line dynamics learning, automatic controller tuning and nonlinear adaptive control [14]-[17]. In our work, we integrate BO with safety requirements and clinical experience, so that the “black-box” glucose regulation process can be safely adapted and improved. The proposed adaptation algorithm features a dynamic parameter selection module that performs performance diagnosis based on glucose data affected by lifestyle disturbances and determines the parameter to be adapted, and a BO-based optimization module that automatically adjusts the selected parameter through learning and optimizing an unknown cost function that quantifies glycemic regulation performance. To integrate clinical experience with the proposed method, a logic-based approach is used in dynamic parameter selection. The proposed parameter adaptation method is evaluated on the 10-patient cohort of the US FDA accepted Universities of Virginia/Padova simulator [18] for two in silico scenarios. We show that for both scenarios and all patients considered, the proposed method is able to correctly identify and adaptively adjust the inappropriate parameters to achieve satisfactory glucose regulation, without causing risks of hypoglycemia throughout the adaptation procedure.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 depicts an overview of an example system to implement an artificial pancreas according to the present disclosure.

FIG. 2 depicts a flow chart of an example method to implement an artificial pancreas according to the present disclosure.

FIG. 3 depicts a flow chart of an example method of parameter adaption according to the present disclosure.

FIG. 4A depicts a chart showing an example of a sequential algorithm according to the present disclosure.

FIG. 4B depicts a chart showing an example of a sequential algorithm according to the present disclosure.

FIGS. 5A-5D depict bar graphs showing various features of the adaption procedure for an in silico subject (Scenario I). FIG. 5A plots the trends of mean BG (day and night), mean BG (overnight) and percent time in [70,180] mg/dL. The daily data points are displayed using colored circles, together with the weekly average and standard deviation of the data points displayed using a colored line and shadow, respectively. Following the same fashion, FIG. 5B displays the percent time below 54 mg/dL and 70 mg/dL for both day and night and overnight. FIG. 5C provides meal information, where the sizes of breakfast, lunch and dinner are displayed in blue, green and yellow, respectively. FIG. 5D shows the parameters used for each week together with the corresponding values; the value of 1=CR (instead of CR) is provided. For illustration purpose, the relative values of the parameters against those at the beginning of the adaptation are provided.

FIGS. 6A-6D depict graphs showing adaptation procedures for the 10-patient cohort (Scenarios I and II). For illustration purpose, the relative values of parameters against those at the beginning of the adaptation are provided. To obtain the mean values, each scenario is simulated for the same amount of time period (24 and 16 weeks for Scenarios I and II, respectively) for all patients; if the adaptation process for a specific patient completes before the end of simulation, the final values of the adaptation parameters are used till the end.

FIGS. 7A-7D depict bar graphs showing various features of the adaption procedure for an in silico subject (Scenario II). FIG. 7A plots the trends of mean BG (day and night), mean BG (overnight) and percent time in [70,180] mg/dL. The daily data points are displayed using colored circles, together with the weekly average and standard deviation of the data points displayed using a colored line and shadow, respectively. Following the same fashion, FIG. 7B displays the percent time below 54 mg/dL and 70 mg/dL for both day and night and overnight. FIG. 7C provides meal information, where the sizes of breakfast, lunch and dinner are displayed in blue, green and yellow, respectively. FIG. 5D shows the parameters used for each week together with the corresponding values; the value of 1=CR (instead of CR) is provided. For illustration purpose, the relative values of the parameters against those at the beginning of the adaptation are provided.

FIG. 8 depicts a flow chart showing an example process for adaptation of feedforward control parameters.

FIGS. 9A-9B depict graphs showing trends of the glycemic management metrics in the adaptation procedures for the 111-patient cohort (Scenario I). A box-and-whisker approach is used to plot the data, where on each box, the central white line is the median, the edges of a box denote the 25% and 75% percentiles, and the whiskers denote the 5% and 95% percentiles.

FIGS. 10A-10C depict graphs showing trends of the adaptation parameters in the adaptation procedures for the 111-patient cohort (Scenario I). The same box-and-whisker approach as that in FIG. 3 is used to plot the data. For illustration purpose, the relative values of parameters against default values in the UVA/Padova simulator are provided.

FIGS. 11A-11B depict graphs showing trends of the glycemic management metrics in the adaptation procedures for the 111-patient cohort (Scenario II). A box-and-whisker approach is used to plot the data, where on each box, the central white line is the median, the edges of a box denote the 25% and 75% percentiles, and the whiskers denote the 5% and 95% percentiles.

FIGS. 12A-12C depict graphs showing trends of the glycemic management metrics in the adaptation procedures for the 111-patient cohort (Scenario II). A box-and-whisker approach is used to plot the data, where on each box, the central white line is the median, the edges of a box denote the 25% and 75% percentiles, and the whiskers denote the 5% and 95% percentiles.

FIGS. 13A-13B depict graphs showing trends of the glycemic management metrics in the adaptation procedures for the 111-patient cohort (Scenario III). A box-and-whisker approach is used to plot the data, where on each box, the central white line is the median, the edges of a box denote the 25% and 75% percentiles, and the whiskers denote the 5% and 95% percentiles.

FIGS. 14A-14B depict graphs showing trends of the glycemic management metrics in the adaptation procedures for the 111-patient cohort (Scenario III). A box-and-whisker approach is used to plot the data, where on each box, the central white line is the median, the edges of a box denote the 25% and 75% percentiles, and the whiskers denote the 5% and 95% percentiles.

FIGS. 15A-15D depict graphs showing twenty-four-hour glucose and insulin profiles simulated using the system parameters obtained on Weeks 1, 8, 16, and 24 of Scenario I for a particular patient. For comparison purpose, the same meal protocol and measurement noise sequence are applied to generate the data in the four subplots.

FIGS. 16A-16D depict graphs showing twenty-four-hour glucose and insulin profiles simulated using the system parameters obtained on Weeks 1, 8, 16, and 24 of Scenario II for a particular patient. For comparison purpose, the same meal protocol and measurement noise sequence are applied to generate the data in the four subplots.

FIGS. 17A-17D depict graphs showing twenty-four-hour glucose and insulin profiles simulated using the system parameters obtained on Weeks 1, 8, 16, and 24 of Scenario III for a particular patient. For comparison purpose, the same meal protocol and measurement noise sequence are applied to generate the data in the four subplots.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Glucose Monitoring and Feedback Control Systems

Patients with T1DM suffer from malfunctions of the glucose metabolic process due to the failure of the pancreas to secrete insulin and require external insulin infusion to regulate excessive blood glucose. An AP takes the role of a healthy pancreas and generates insulin micro-boluses according to the trends and changes of blood glucose level.

The human glucose metabolic process, however, is affected by disturbances that occur under different time scales, including 1) food intakes, physical exercises and alcohol consumptions occurring on a random basis, 2) the diurnal circadian rhythm of the body sensitivity to insulin and life habits that repeat on a daily/weekly basis and 3) chronic metabolic variations due to aging and lifestyle change.

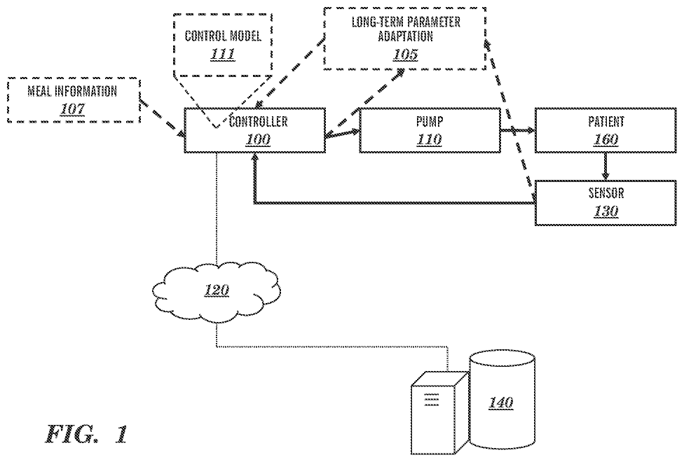

To achieve satisfactory glucose regulation performance, all these disturbances need to be considered in the design of glucose control algorithms. Considering the multi-timescale nature of the disturbances, we introduce a dual-layer control scheme in this invention (see FIG. 1). The lower layer is composed of multiple controllers that deal with short-term disturbances, including:

(1) a basal rate calculator that provides the basal insulin delivery rate that is pre-determined according to patient's life habit and the diurnal insulin sensitivity;
(2) a feedforward controller that calculates meal and correction boluses based on meal information and additional insulin requests provided by users; and
(3) a feedback controller that deals with all uncompensated disturbances based on real-time CGM measurements and safety consideration.

In some examples, the feedback controller tops up the basal rate doses provided by the basal rate calculator to finalize real-time insulin micro-boluses. With a properly designed basal rate profile, the feedback controller does not need to act with drastically changing and scenario-dependent aggressiveness, which reduces the difficulty of feedback control design. Feedforward control (e.g., meal boluses) can timely reduce high-glucose events and is therefore important in glucose management, particularly when meals cannot be accurately predicted. Lower-layer control system design has been extensively investigated in the AP literature, the focus of which has been developing safe and efficient feedback control algorithms to improve glucose management; interested readers can refer to [19] for a detailed introduction of state-of-the-art developments.

The upper layer is responsible for long-term parameter adaptation of lower layer control algorithms. This layer evolves on a longer timescale (e.g., weeks) and handles chronic changes in the patient's glucose metabolic process and life style based on the historical performance metrics. Long-term AP adaptation has gained its importance only recently, with the successful deployment of large and long-term out-patient clinical studies, and is the main problem considered in this invention. We focus on how to automatically and correctly identify the parameters to be adapted and how to safely and efficiently optimize the identified parameters based on historical data. To solve these problems, we introduce a BO-based controller adaptation framework in this invention.

FIG. 1 illustrates an overview of an example system for implementing the disclosed technology. For instance, the system includes a controller 100 that provides instructions to a pump 110 to provide insulin boluses to a patient 160. The controller 110 may include a control system that has one or more processors, memory and may include one or more control models 111 stored on a memory that process glucose data output from a sensor 130, meal information 107, and other data to determine a bolus size of insulin that needs to be delivered to the patient 160 and sends the instructions to the pump 110.

The controller 110 may be in communication with the pump by a wired or wireless connection. Additionally, the glucose sensor 130 may be any suitable sensor for continuous glucose monitoring, and may be an under the skin sensor with a wireless connection to the controller 100. In other examples, it may be a non-invasive sensor 130 and have a wired or wireless connection to the controller 100, for instance the FreeStyle Libre manufactured by Abbott Laboratories.

The pump may be any suitable insulin pump that is capable of receiving instructions from the controller 100 and delivering insulin boluses to the patient 160. For instance, the Medtronic MiniMed 670G is an artificial pancreas using a closed-loop system that includes an insulin pump.

The controller 100 may include models for long term parameter adaptation 105 as described herein. This may include adaptation of a basal rate profile of a patient and the carbohydrate ratio that may be altered through lifestyle changes.

The controller 100 may also be connected over a network 120 to a server 150 and a database 140. In some examples, various calculations and model processing will be carried out on local processors on the controller 100 and save on local memory. In other examples, the calculations could be carried out on a server 150 or other computing device in communication with the controller 100.

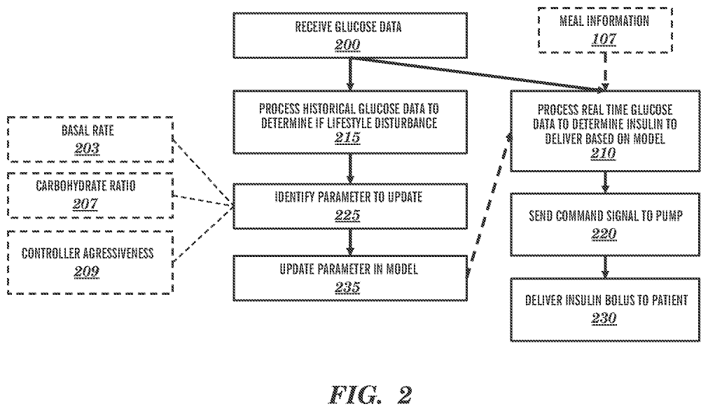

FIG. 2 illustrates an example method for implementing the presently disclosed technology. For instance, a controller 100 or other control system may receive glucose data 200 output from a glucose sensor 130. The received data may be periodically received, relatively continuously received, daily received, or other suitable time periods of measurements. In some examples, the glucose level data will be stored in a memory in the controller 100 or a database 140.

In some examples, the glucose level data will be stored and analyzed during certain time windows. For instance, the real time glucose data may be processed by the controller 100 and associated control model 111 to determine a real time bolus of insulin to deliver 210. This may be performed using a control relevant model as disclosed herein according to certain parameters. Once the bolus size is determined, a command signal may be sent to a pump 220 and the pump would then deliver a bolus of insulin to the patient 230.

Additionally, high level adaptation of long term parameters may take place by processing historical glucose data to determine if there is a lifestyle disturbance 215 that requires updating parameters of the control model 111. In some examples, this may include a previous time window of glucose readings, for instance 1 day, a couple days, one week, two weeks, a month or other suitable time periods.

In some examples, different windows of time during the data may be analyzed over longer time periods. For instance, the average fasting or nighttime blood glucose level may be analyzed to determine whether the basal rate parameter 203 needs to be updated by averaging glucose levels from 10 pm to 6 am every day for a week. In other examples, the average (or other statistical metric) glucose levels may be monitored after a meal bolus of insulin is delivered over a week or other time period to determine whether the carbohydrate ratio parameters 207 need to be updated. Additionally, all time periods may be monitored to determine whether controller aggressiveness parameters 209 need to be updated.

In these examples, the system may then determine if a parameter needs to be updated and which parameter to update 225. For instance, if the fasting blood glucose is outside of a desired or pre-defined threshold range, it may be concluded that the basal rate of the patient has changed due to a lifestyle disturbance. Accordingly, the system may identify the basal rate parameter 203 to be updated.

Next, the system may update the basal rate parameter in the model 235. This may be performed through a variety of methods, for instance by performing a Bayesian optimization model as disclosed herein. Other suitable methods could be utilized as well using historical glucose data. Additionally, once this procedure is performed, the control model 111 parameters will have been updated and the real time glucose may be processed with new parameters and the updated model 210.

The process may iteratively continue until the analysis of the historical glucose data indicates that the lifestyle related parameters no longer need updating because the relevant glucose data metrics are within predefined thresholds. Then, the system may continually check the historical data until another disturbance is detected 215.

### EXAMPLES

The following examples are provided to better illustrate the claimed invention and are not intended to be interpreted as limiting the scope of the invention. To the extent that specific materials or steps are mentioned, it is merely for purposes of illustration and is not intended to limit the invention. One skilled in the art may develop equivalent means or reactants without the exercise of inventive capacity and without departing from the scope of the invention.

### Example 1: Bayesian Based Optimization Based Controller Adaptation Framework

Following is an example implementation of the two level framework for AP controller design. In this example, the lower layer control tasks may be handled by the periodic zone model predictive control (MPC) with asymmetric costs (with a 5-minute sampling time) [20] together with the meal bolus strategy introduced therein, although the proposed approach can be applied to other control algorithms.

To aid the subsequent description, Φ denotes the set of parameters to be adapted and use Φₖ to represent the set of variables corresponding to the parameters in at iteration k of the adaptation process. Roughly Φ is a set of the names of the parameters while k denotes the actual set of parameters with values. Similarly, ϕ denotes an element in and ϕₖ to represent the value of ϕ at iteration k. Basically, Φ can be composed of various parameters in the basal rate calculator, feedforward meal bolus controller and feedback controller (e.g., nominal basal rate, carbohydrate ratio, penalty matrices in the MPC cost function, and parameters of the insulin-on-board constraints).

In this disclosure, it is assumed that assume Φ:={β, CR, \hat{R}}, where β denotes the nominal basal rate profile, CR denotes CHO ratio profile and \hat{R} is the control penalty parameter that determines the insulin delivery above basal rate in the cost function of the zone MPC [20]. The reason of choosing these parameters is that β and CR profiles decide the performance of the “open-loop” basal rate calculator and meal bolus controller, while \hat{R} controls the aggressiveness of the MPC used [20]; CR and β were used as adaptation parameters in a recent clinical study [4], in which the CHO ratio and total basal profiles were changed by as much as 20% and 2.5 units/day, respectively. Correspondingly, Φₖ:={βₖ, CRₖ, \hat{R}ₖ}.

As insulin sensitivity is assumed to be constant in the simulation environment [18] used in this example, it is assumed that basal rate and CHO ratio are constant in a day and thus βₖ and CRₖ are scalars. This simplification is made based on the fact that some patients use a single value for CHO ratio across the day and basal rate that clinically is a single value. The proposed approach, however, can be extended to consider general basal rate and CHO ratio profiles. In general, parameter adaptation can be formulated into a constrained optimization problem

min_{ϕ}_{ₖ}ƒ(ϕₖ)

s.t., g(ϕₖ)≤0, (1)

where ƒ(Φₖ) denotes the objective function that represents glucose regulation performance (e.g., average glucose level), and g(ϕₖ)≤0 represents the safety constraints that restrict the severeness of hypoglycemia.

To cope with chronic metabolic variations and long-term lifestyle changes, the upper-layer parameter adaptation algorithm should be able to automatically diagnose the root cause of unsatisfactory glycemic regulation performance through exploiting historical data, intelligently map the identified root cause with the appropriate tuning parameter in the lower-layer algorithms, and optimize the parameter efficiently and safely towards the long-term goals of glucose management within an acceptable length of time period, without causing hypoglycemia risks during the adaptation process. The analytical and quantitative relationship between the performance metrics of glucose management and the candidate parameters in the lower-layer control algorithms, however, is generally unknown.

In this example, Bayesian Optimization (“BO”) appears to be an intriguing approach, which was recently developed by researchers in machine learning [13] as a powerful tool to solve optimization problems with unknown objective functions. In particular, the Bayesian nature of the approach improves data efficiency, which helps ensure the speed and effectiveness of parameter adaptation. The safety requirements in AP design, however, hamper the direct adoption of BO method for AP adaptation at home, which is an important challenge to overcome.

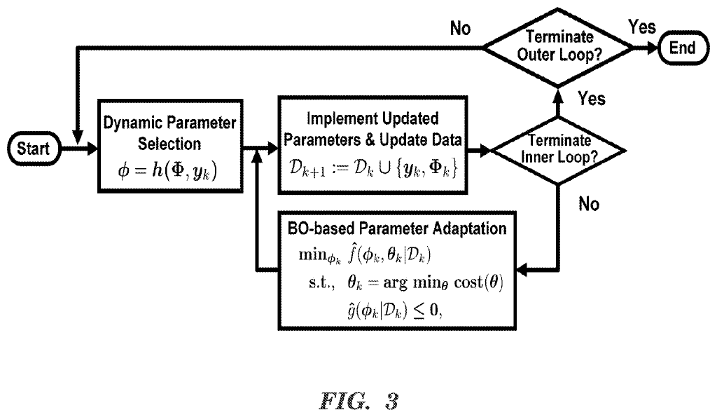

Based on the above discussion, disclosed is a “hybrid” dual-loop parameter optimization framework that merges the features of BO with clinical knowledge and safety constraints; a schematic of the proposed framework is provided in FIG. 3. As the analytical expressions of ƒ(⋅) and g(⋅) are not known, the parameter adaptation problem may be solved in a data-driven fashion, and simplify the problem in into an accessible form:

min_{ϕ}_{ₖ}\hat{ƒ}(ϕₖ,θₖ|Dₖ)

s.t., θₖ=arg min_{θ}Σ_{t∈π}_{ₖ}(ƒ(Φₜ)−\hat{ƒ}(ϕₜ,θ|Dₖ))²,

ĝ(ϕₖ|Dₖ)≤0,

where ϕₖ ∈Φₖ denotes a tuning variable selected through

ϕ=h(ϕ,yₖ)

In this example, ϕₖ denotes the value of ϕ. Here, k counts the iteration of the adaptation process, which is assumed to have a larger timescale (e.g., days) compared with the sampling time of the lower layer algorithms, πₖ denotes the set of all iteration indexes for which Φₜ\{ϕₜ}=Φₖ\{ϕₖ} hold for t∈πₖ, Dₖ denotes the amount of data available at iteration k before performing adaptation, satisfying

Dₖ₊₁=Dₖ∪{yₖ,Φₖ}

Note that yₖ here denotes the glucose data sequence obtained in iteration k using Φₖ and is assumed to contain CGM data of n days; to make this point clear, we write

yₖ:={y_{k,1},y_{k,2}, . . . ,y_{k,n}},

where y_{k,i} denotes the CGM data on day i. For completeness, D₀:=Ø, denote Φ₀ as the set of initial tuning parameters setting and let y₀ denote data before performing adaptation. Additionally, Dₖ⊆Dₖ as the subset of data corresponding to iteration indexes in πₖ. In the above equations, one can replace ƒ(⋅) and g(⋅) with their data-driven delegates \hat{ƒ}(⋅) and ĝ(⋅); in particular, an internal optimization problem is solved to estimate Ok based on the available data Dₖ, so that \hat{ƒ}(⋅) becomes available for the minimization of ϕₖ. In particular, as ƒ(Φₜ) is measurable for t≤k, \hat{ƒ}(⋅) can be estimated through solving a convex optimization problem if the structure of \hat{ƒ}(⋅) is suitably selected.

For safety consideration, only one parameter is adjusted at each iteration k; thus ϕₖ is a scalar presenting the value of one of the tuning parameters in Φ. An equality constraint is utilized to determine the parameter ϕ to adapt at iteration k, which is decoupled from the optimization problem; the consideration of this constraint allows the integration of clinical knowledge into the optimization problem, as will be shown later.

In the dual-loop optimization framework, the design of h(Φ, yₖ) is handled in the outer loop, which performs the task of dynamic tuning-parameter selection, while the optimization problem is approached via BO in the inner loop, based on the ϕ selected by the outer loop.

### Dynamic Parameter Selection

Although BO offers the flexibility of multivariate optimization, simultaneous adjustment of multiple tuning parameters is not explored in the proposed long-term AP adaptation for a couple of reasons. Firstly, clinical experience indicates that diabetic symptoms can be directly associated with certain parameters; for instance, frequent postprandial hyperglycemia can be attributed to improper meal bolus sizes. Secondly, the selected tuning parameters can have coupling effects on the performance metrics, the joint adjustment of which can add to the difficulty of understanding the correct direction of parameter adaptation. Thirdly, the goal of parameter adaptation is to achieve satisfactory rather than optimal glucose management. Indeed, it would be preferable to have an algorithm that safely achieves satisfactory metrics within a smaller number of iterations, as each iteration may take weeks for data collection to average out the effect of lifestyle, compared with one that identifies the optimal solution with a much larger number of iterations and even increased risks of hypoglycemia. On the basis of the above discussion, only one tuning parameter is adapted at each iteration k; the outer loop of our adaptation framework is designed to determine the suitable tuning parameter ϕ∈Φ.

The outer loop is composed of a dynamic parameter selection module and a terminal condition module. The dynamic parameter selection module implements the equality constraint ϕ=h(Φ, yₖ) in (5). The design of h(Φ, yₖ) builds on the idea of constructing a map from a list of symptoms to Φ, which incorporates clinical experience into the proposed adaptation framework. In this work, we consider three classes of symptoms: overnight (24:00-06:00) hyper/hypoglycemia, postprandial hyper/hypoglycemia, and overall hyper/hypoglycemia; here “overall” means day and night. Similar to [12], we assume that glucose responses are dominated by the basal infusion rate at night, as meals usually do not take place between 24:00 and 06:00. Postprandial glucose metrics are mainly affected by meal boluses, the size of which is adjusted by CR.

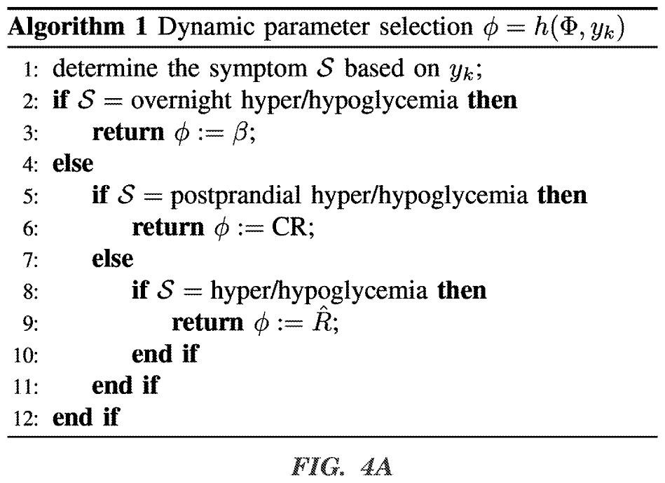

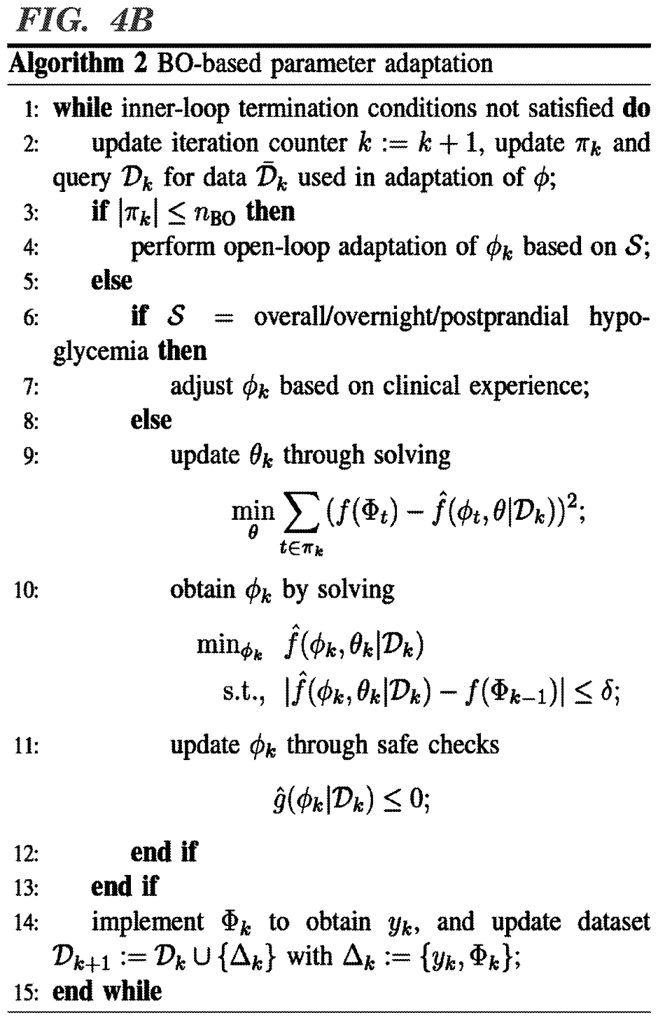

Finally, if the nighttime and postprandial metrics are satisfactory but the subject still suffers from hyperglycemia or hypoglycemia, the controller parameter \hat{R} would be adjusted. With these principles, an algorithmic description of h(Φ, yₖ) is provided in Algorithm 1 shown in FIG. 4A, which allows the solution of the multivariate adaptation problem in a fashion similar to hierarchal optimization.

In this algorithm, overnight issues are given the highest priority as hypoglycemia and hyperglycemia that happen when the patient is asleep have greater risks. As meals are usually the major disturbances to glucose control, postprandial performance is also given a higher priority. Note that Algorithm 1 in FIG. 4A provides a simple embodiment of selecting the tuning parameter based on clinical experience; more sophisticated designs are possible if more classes of symptoms and tuning parameters are considered.

The terminal condition module in the outer loop determines whether or not to stop the adaptation process. Two conditions are considered. The first condition checks whether all the symptoms have disappeared as the result of adaptation. The second condition deals with the case that the goal of eliminating the symptoms is unachievable through adaptation, which is realistic as the symptoms are defined based on user-specified parameters. To deal with this situation, one can introduce the maximum allowable times that each of the tuning parameters is to allowed to be selected; this would force the adaptation procedure to stop if the goals are not achievable after some iterations.

### BO-Based Parameter Adaptation

The inner loop of the adaptation scheme optimizes ϕₖ, which represents the value of the selected parameter ϕ, based on the available data Dₖ. This is done through solving the equations above. In the BO literature, different forms of \hat{ƒ}(ϕₖ, θₖ|Dₖ) have been proposed [13]. As approximate linear relationships between the average glucose level and adaptation parameters are observed in the in silico studies, a linear kernel is adopted in this work:

\hat{ƒ}(ϕₖ,θₖ|Dₖ):=θ_{k,1}ϕₖ+θ_{k,2}

with θₖ:=[θ_{k,1}, θ_{k,2}]^{T}. Note that to make efficient use of data, the value of θₖ is made ϕ-dependent and is obtained based on the segment D̅ₖ⊆Dₖ for which the current ϕ is adapted while the values of k/{ϕₖ} are kept constant. This further explains the reason of using a linear kernel: the cost function simply represents a local linearization of the unknown cost function ƒ(Φₖ) around the adopted values of k. The performance metric represented by the objective function is also parameter-dependent in the adaptation procedure; if basal rate β is adapted, ƒ(⋅) denotes average blood glucose overnight, otherwise ƒ(⋅) represents average glucose throughout day and night. Considering the risk sensitive nature of the adaption problem, the hypoglycemia risk constraints in (4) are implemented as

ϕ(D̅ₖ)≤ϕₖϕ̅(D̅ₖ)

which provide limitations for ϕₖ to avoid severe hypoglycemia. The values of ϕ(D̅ₖ) and ϕ̅(D̅ₖ) a simple data-driven fashion through querying D̅ₖ.

To solve (2)-(4), a BO-based algorithm is proposed (see Algorithm 2 in FIG. 4B), which iteratively adapts tuning parameter Ok until the inner-loop terminal conditions are satisfied. For each iteration, the algorithm starts with updating the value of ϕₖ according to its definition, and obtains D̅ₖ ⊆Dₖ based on πₖ (line 2 of the algorithm). If either there is not enough data (namely, |πₖ|≤n_{BO}) to perform data fitting (line 3) or the subject is diagnosed with risk of hypoglycemia (line 6), heuristic adjustment of ϕₖ based on clinical experience will be performed for safety concern. As a linear kernel is used in our work, n_{BO} is chosen to be 2.

For all other scenarios, ϕₖ is adjusted through the proposed BO procedure (lines 9-11). The BO first estimates θₖ based on D̅ₖ (line 9). With the obtained θₖ, ϕₖ is calculated by solving a constrained optimization problem (line 10), where a constraint |\hat{ƒ}(ϕₖ, θₖ|Dₖ)−ƒ(Φk)|≤δ is added for safety concern to ensure the smoothness of the adaptation process; in this implementation, δ is chosen to be 4 mg/dL to allow smooth and timely adaptation. As \hat{ƒ}(ϕₖ, θₖ|Dₖ) is linear with respect to Ok, the optimization problem is actually a linear programming problem. Safety checks are further performed for ϕₖ (line 11), to avoid the risk of hypoglycemia. The obtained Ok is then implemented on the patient for a certain period of time (which is assumed to be 1 week in this work), during which the glucose data yₖ is collected to update Dₖ (line 14).

Four types of inner-loop terminal conditions are considered. The first condition is that the current symptom S has been eliminated through adaptation. The second condition is that a different symptom is caused by the adaptation process. The third condition is that the tuning parameter no longer changes, which is measured in terms of |ϕₖ−ϕₖ₋₁|. The last conditions is that a large change of the current parameter does not change the concerned performance metric. In particular, the second and fourth conditions handle the case that a wrong parameter is selected by the outer loop, which is unavoidable as the metrics of glycemic control are subjected to lifestyle disturbances. The first and third conditions are similar to terminal conditions adopted in standard optimization algorithms. The inner loop will be terminated if one of these conditions are met.

### Data-Driven Robust Symptom Diagnosis

One can focus on the determination of symptom S based on glucose data yₖ, which corresponds to line 1 of Algorithm 1 in FIG. 4A. This is of crucial importance to the success of long-term adaption, since the proposed parameter adaptation procedure is performed when the subjects are in free-living environments and disturbances caused by lifestyle are inevitable. In this regard, a data-driven “symptom” decision method that is robust to lifestyle disturbances should be proposed.

In this example, 6 types of symptoms are considered, including overnight (24:00-06:00) hyper/hypoglycemia, postprandial hyper/hypoglycemia, and overall hyper/hypoglycemia. To aid the development of the adaptation algorithm, S is assigned one of these symptoms or Ø when no symptom is diagnosed. As a subject may have multiple symptoms at the same time, one can assign different priorities to the symptoms and the symptom with highest priority is assigned to S. Specifically, one can give overnight hyper/hypoglycemia the highest priority, overall hyper/hypoglycemia the lowest priority, and postprandial hyper/hypoglycemia the intermediate priority.

To implement the proposed adaptation scheme, overnight hyper/hypoglycemia is determined based on glucose data between 24:00 and 06:00 in {yₖ,i}, postprandial hyper/hypoglycemia is evaluated based on CGM readings 3.5 hours after an announced meal, and overall hyper/hypoglycemia is evaluated based on all glucose data in {yₖ,i}.

To ensure robust diagnosis of overnight and overall hypoglycemia, one can calculate the percent time below 54 mg/dL pct54_{k,i}, percent time below 70 mg/dL pct70_{k,i}, percent time below 54 mg/dL at night pct54N_{k,i}, and percent time below 70 mg/dL at night pct70N_{k,i} based on each yₖ, i∈yₖ. With the obtained data sequences, one can perform right-tailed Wilcoxon signed rank tests [21] to evaluate if the sequences {pct54_{k,i}}, {pct70_{k,i}}, {pct54N_{k,i}}, {pct70N_{k,i}} are obviously larger than pre-specified thresholds, which helps rule out lifestyle disturbances.

In this implementation, the thresholds are chosen as 0%, 2%, 0% and 0%, respectively. In addition, if the overnight/overall mean glucose are below user-specified thresholds (125 mg/dL and 115 mg/dL in our implementation), overnight/overall hypoglycemia will be diagnosed as well. Overnight and overall hyperglycemia are diagnosed if the average glucose levels within the concerned time periods exceed pre-specified thresholds (which are chosen as 140 mg/dL and 135 mg/dL in our implementation, respectively). Postprandial hyperglycemia and hypoglycemia are determined through testing if average glucose level 3.5 hours after a meal is greater/less than user-specified thresholds, which are chosen as 140 mg/dL and 120 mg/dL in our implementation. Note, however, that different methods of defining and diagnosing the symptoms and different choice of the thresholds can be considered, which will not affect the inner- and outer-loop adaptation algorithms proposed.

### In Silico Results

In this example, the proposed adaptation algorithm was evaluated through performing multiple-month simulations on the 10-patient cohort of the US FDA accepted Universities of Virginia/Padova simulator [18]. To mimic lifestyle disturbances, the in silico subjects take breakfast, lunch and dinner with normally distributed meal sizes (with means and standard deviations equal to [50, 65, 65] g and [8, 8, 8] g CHO) and meal times uniformly distributed in [07:00, 09:00], [11:00, 13:00] and [18:00, 20:00], respectively; in addition, each meal can be skipped with probability 0.1. The CGM measurement noise is generated according to a random noise seed on each day. It was assumed that the meals are all fully announced but the meal boluses are calculated with potentially inappropriate CR. The updated parameters obtained in each iteration are used for 1 week (7 days) [10] before the next iteration, so that enough data can be collected for performance evaluation and diagnosis.

To evaluate the safety, effectiveness, and robustness of the adaptation algorithm, two scenarios are considered. In the first scenario (Scenario I), the patients are assumed to have doubled CHO ratio and halved basal rate, both of which will lead to increased hyperglycemia due to conservative insulin delivery. In the second scenario (Scenario II), the patients are initiated with doubled CHO ratio and doubled basal rate; the former would cause conservative meal boluses but the latter would counteract with relatively larger insulin micro-boluses, which makes it challenging for the adaptation algorithm to identify the appropriate tuning parameters.

For all in silico subjects, it was assumed that the zone MPC developed in [20] with default parameters was used, together with constant basal rate profiles. The parameters in the adaptation algorithm are specified herein. For both scenarios, the first week is utilized to collect data for initialization and thus no parameter is selected and adjusted; the adaptation process starts from the second week. The performance metrics are selected according to [22].

### Results for Scenario I

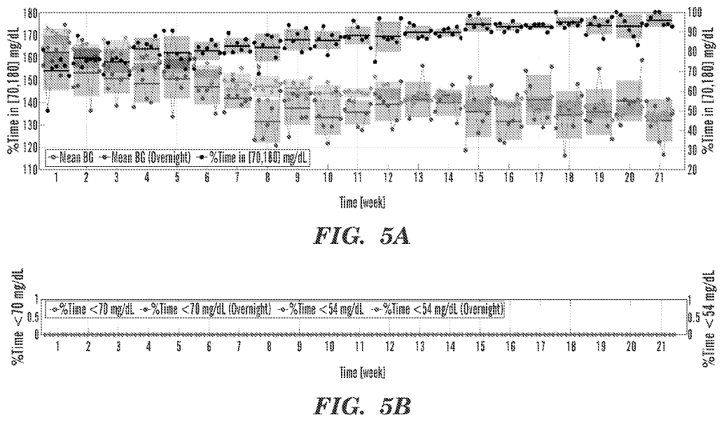

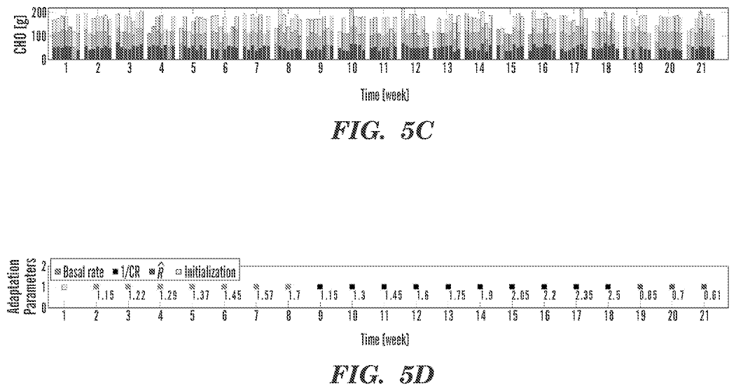

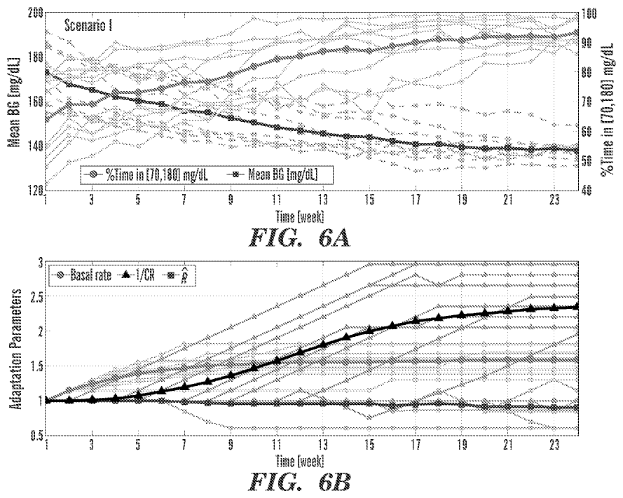

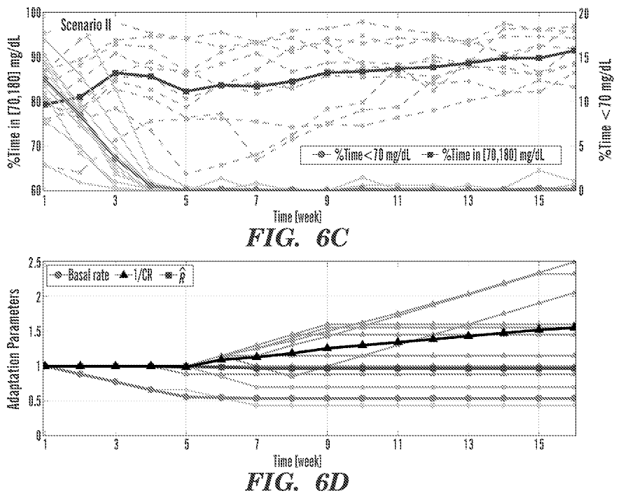

Results for Scenario I are shown in FIGS. 5A-5C and 6A, and 6B. FIGS. 5A-5D provide the adaptation procedure of a subject in the 10-patient cohort. As expected, the patient has high average glucose and low percent time in [70,180] mg/dL at the beginning of the simulation, due to the small meal boluses and basal rates. One can observe that the adaptation algorithm is able to identify the correct tuning parameters (β and CR) with the dynamic parameter selection module and adjust the parameters towards to correct direction for improved glucose regulation performance, despite the disturbances caused by randomized meal sizes and time (see the variations in the daily average BG in FIG. 5A). To achieve the goals of adaptation, the aggressiveness of closed-loop control is also adjusted a bit through adapting \hat{R}. The adaptation algorithm is safe in the sense that no obvious hypoglycemia risk is caused during the process.

Adaptation performance for the entire 10-patient cohort is shown in FIGS. 5A and 5B. For all patients the adaptation algorithm manages to improve glycemic control performance dramatically in terms of both average glucose levels (from 173.1 mg/dL (week 1) to 138.0 mg/dL (week 24); p<0.001) and percent time in euglycemia range [70,180] mg/dL (from 63.9% to 93.2%; p<0.001). Although not displayed in the figure, no hypoglycemia risk is caused by the algorithm for all patients. Also, one can observe that population-wise the algorithm is able to increase basal rates and decrease carbohydrate ratio, while the values of \hat{R} are generally unchanged. Convergence is observed with minimal changes in the parameters for most patients after week 23.

### Results for Scenario II

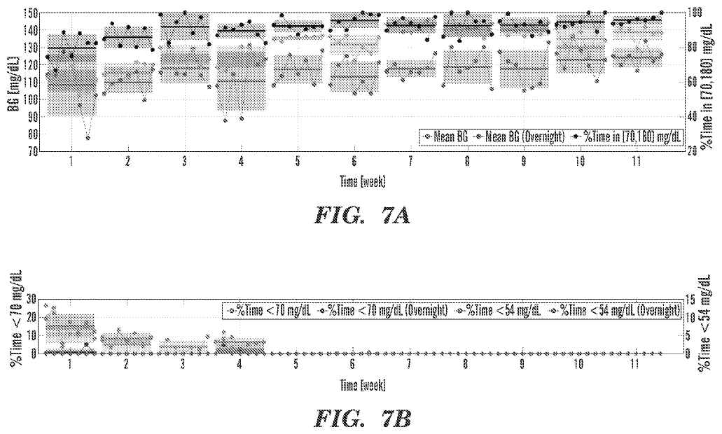

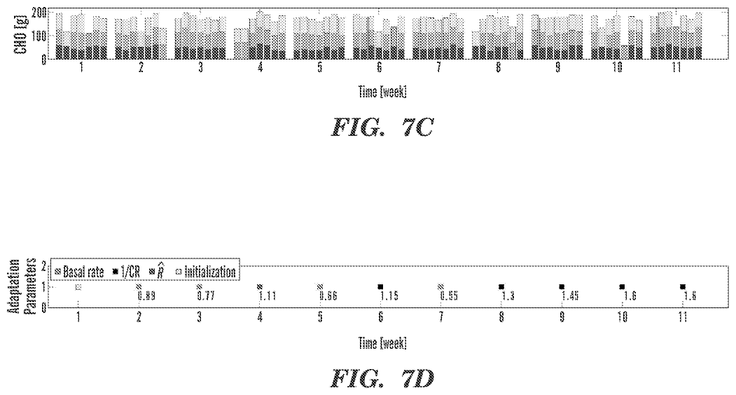

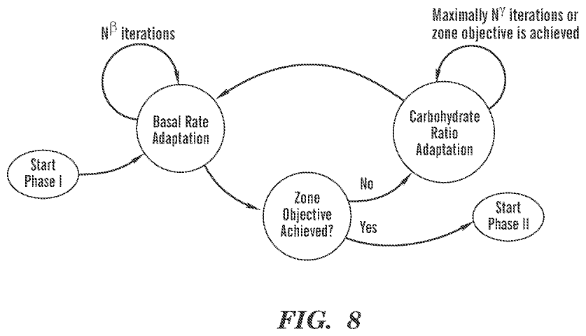

Results for Scenario II are provided in FIGS. 7A-7D and FIG. 6C—6D. FIG. 7 provides the adaptation procedure of an in silico patient. One can observe that the adaptation algorithm is able to adjust the parameters (β and CR) towards the correct directions. Note that the activeness of closed-loop control is reduced on week 4, as feedback control is diagnosed as the cause of hypoglycemia risk (due to life style disturbances), but from week 5 it continues to adjust basal rate which is the exact cause.

Results for the entire 10-patient cohort are shown in FIG. 6C-6D. One observe that the proposed adaptation approach is able to eliminate hypoglycemia induced by overestimated basal rates (percent time below 70 mg/dL, from 12.5% (week 1) to 0.2% (week 16); p<0.001) and improve percent time in [70,180] mg/dL (from 79.4% to 91.4%; p<0.001), through decreasing basal rate and carbohydrate ratio with statistical significance while in general keeping \hat{R} un-adjusted. For this scenario, convergence is observed for most patients after week 15, with minimal further parameter changes.

### Example 2: Bayesian Based Optimization Based Controller Adaptation Framework

In this example, another implementation and in silico testing of the disclosed technology is disclosed. In this example of the dual-layer control scheme, the parameters to be adapted include:
1. segments {βₙ} that compose the BR profile β:=[β₁, β₂, . . . , βₖ] used in the BR calculator. Note that each βₙ is associated with a time period Tₙ^{β} during which it is active. In this example, it is assumed that Tₙ^{β} is fixed and predetermined by clinicians.
2. segments {γₙ} that compose the CR profile γ:=[γ₁, γ₂, . . . , γₘ] used in the meal controller. Similar to β, each segment γₙ is used for a particular time period Tₙ^{γ}, which is assumed to be fixed by clinicians as well.
3. parameters in the closed-loop controller. Three parameters in the zone MPC developed in Reference 20 are considered, including \hat{R} that represents the control penalty parameter for insulin delivery above the BR, D̅ that determines the upper bound of a glucose zone for which the velocity penalty is active, and a coefficient γ_{IOB} that determines the responsiveness of the insulin-on-board (IOB) constraint.

In this example, an automatic parameter learning algorithm is disclosed that can correctly adapt the parameters in the lower-layer control algorithms and is robust to lifestyle disturbances, with minimal patient/clinician involvement and without causing risks of hypoglycemia during the adaptation procedure.

To achieve this goal, a data-driven multivariate parameter learning framework is proposed. Considering the different roles of the lower-layer control algorithms, the adaptation procedure is divided into two phases. In Phase I, the parameters in the feed forward control algorithms (namely, the BR and CR profiles) are optimized. Based on the obtained/updated BR and CR profiles, parameters in the feedback control algorithms are adjusted in Phase II. In both phases, one can consider the challenging but realistic case that the patient is under closed-loop control, such that the “open-loop” parameters are adjusted in Phase I based on closed-loop data. The detailed adaptation algorithms are presented below.

### Learning Feedforward Control Parameters

In this example, Phase I of the adaptation procedure is adapting the parameters in the BR and CR profiles. The aim here is to obtain reasonable rather than optimal profiles, such that an appropriate operating point is provided for the feedback controller, which helps enhance the safety of closed-loop glucose control. More importantly, considering the fact that the feedback control may not be available for some periods (e.g., when the controller runs out of battery or lost CGM connection), the obtained parameter needs to be “safe” in the sense that no hypoglycemia would be caused when the patient loses closed-loop control. In the following, one can first separately introduce the methods for BR and CR adaptation, based on which a hybrid time- and event-triggered method is introduced to iterate the procedures that adapt BR and CR.

### Adaptation of BR Profile

Intuitively, the BR profile β is designed to manage “healthy” fasting glucose levels without considering meal-induced glucose excursions. However, when the fasting glucose levels (measured as glucose levels at night or before meals) are not satisfactory, it is difficult to diagnose which segment βₙ in β is the root cause. This problem becomes more complicated when the effects of meal boluses are considered, due to a further loss of “identifiability.”²¹ A helpful observation, however, is that the “effective” real-time BR is ultimately determined by the feedback controller, which is able to adjust a potentially inappropriate BR provided by the BR profile to a certain extent.

This observation allows one to perform BR profile adaptation using a controller-led approach, without the need of explicitly performing root cause diagnosis. To reduce the risk of controller-induced hypoglycemia, safety constraints are designed to eliminate the adoption of aggressive BR profiles. Through controller-led BR adaptation, the problem of lack of identifiability/diagnosability can be automatically solved, which leads to a direct separation of the adaptation of BR and CR profiles. The difficulty of algorithm design and implementation, however, is independent of the number of segments in the BR profile.

### Controller-Led BR Profile Update

BR adaptation determines the values of BR segments {βₙ^{k+1}} at iteration k+1 based on {βₙ^{k}} and available glucose and insulin delivery information. The idea is to update the BR segment fin with the averaged non-meal related insulin microboluses commanded by the feedback controller during the same time interval, namely, Tₙ^{β}. Although different methods can be used to distinguish meal/non-meal related insulin, one can consider a simple approach in this work and define non-meal related insulin as the insulin deliveries that happen τₘ hours after the previous meal. This defines a set of admissible meal-related insulin deliveries Iₙ^{d} for each BR segment βₙ:

Iₙ^{d}:={(t,Iₜ^{d})|t∈Tₙ^{β},t≥Tₘ^{d}(t)+τₘ}, (1)

where Iₜ^{d} denotes an insulin delivery at time instant t on day d, Tₙ^{β} denotes the time period for which βₙ is active, and Tₘ^{d} (t) denotes the time of the previous meal that happens before time t on day d. We take τₘ=3 hr in our implementation. An initial BR estimate β̅ₙ^{k} for βₙ^{k} can be written as

$${\overline{β}}_{n}^{k} = \frac{{\sum }_{d = 1}^{{n}_{w}} {\sum }_{t = 1}^{{n}_{d}} {I}_{t}^{d} \cdot 1 ( ( t , {I}_{t}^{d} ) ∈ {I}_{n}^{d} )}{{\sum }_{d = 1}^{{n}_{w}} {\sum }_{t = 1}^{{n}_{d}} {I}_{t}^{d} 1 ( ( t , {I}_{t}^{d} ) ∈ {I}_{n}^{d} )} , \qquad ( 2 )$$

where n_{d} denotes the number of insulin microboluses in a day, and n_{w} denotes the number of days in iteration k of the adaptation process, and 1(.) denotes the indicator function. Although the actual real-time BR will be decided by the feedback control algorithm (using the knowledge of βₙ), the key consideration on the obtained βₙ^{k} is that it should not overestimate the “true” BR and cause hypoglycemic events, which is particularly important when the feedback controller is unavailable. To address this concern, two types of safety constraints are incorporated, as will be introduced below.

### Statistical IOB Constraint

Similar to the case with lower-layer control algorithms, the IOB information can be exploited to prevent aggressive behavior of the parameter adaptation algorithm. On the basis of the IOB calculation used in zone MPC,²⁰ a statistical IOB constraint is proposed for BR adaptation. Specifically, a dynamic database D(βₙ) is built to eliminate overestimated values of βₙ, which stores the information of a triplet {IOBₙⁱ, βₙⁱγ_{m˜n}ⁱ} that have led to low-glucose events in history for i∈{1,2, . . . , k}, with IOBₙⁱ denoting the averaged IOB value immediately before βₙ becomes active in adaptation iteration i, and γ_{m˜n}ⁱ denotes the values CR used within Tₙ^{β} iteration k. When βₙ^{k+1} is computed for iteration k+1 and β̅ₙ^{k+1}>βₙ^{k}, the IOB constraint is enforced and implemented as

[IOBₙ^{k+1},β̅ₙ^{k+1},1/γ_{m˜n}^{k}]≤[IOBₙⁱ,0.95βₙⁱ,1/γ_{m˜n}ⁱ] (3)

for all {IOBₙⁱ, βₙⁱ, γ_{m˜n}ⁱ}∈D(βₙ), where “≤” denotes an element-wise partial order holds for the two vectors compared. Here, IOBₙ^{k} is used to estimate the average IOB value immediately before βₙ becomes active in adaptation iteration k+1. If any of the IOB constraints are violated, βₙ^{k+1}=0.95βₙ^{k} to avoid causing hypoglycemia risk.

### Smoothness Constraint

This constraint ensures that the BR segment βₙ does not change too much compared with its neighboring segments βₙ₋ and βₙ₊, with n⁻=N−mod(N−n+1, N) and n⁺=mod(n, N)+1, respectively. The underlying intuition is that the insulin requirement of the metabolic system to maintain a healthy fasting glucose level does not change abruptly in time. Mathematically, this constraint is implemented as

βₙ^{k+1}=min(β̅ₙ^{k+1},λₛ^{β} min(β̅ₙ_{^{−}}^{k+1},β̅ₙ_{⁺}^{k+1},βₙ_{^{−}}^{k},βₙ_{⁺}^{k})),

where λₛ^{β} denotes the “smoothness coefficient” and is selected as 1.3 in this example to compromise smoothness with performance.

### Adaptation of CR Profile

Different from BR adaptation, the effect of different CR segments on glucose profiles are relatively isolated, as meals are usually several hours apart. This helps decouple the adaptation problems for different γₙ values, for which a data-driven optimization approach can be developed.

Instead of optimizing the CR profile for a specific glycemic metric, the goal of this example for CR adaptation is to only ensure that the average BG levels {yₙ^{γ}|n=1, 2, . . . , M} after meals are taken for τ_{y} hours should settle within a certain zone [y̅^{y},y^{y}]; in this implementation, this zone is meal-dependent and is selected as [125, 155], [135, 165], and [125, 155] mg/dl for breakfast, lunch, and dinner, respectively, based on simulation data from the UVA/Padova simulator.

On one hand, this choice of a control-to-range objective helps efficiently handle the uncertainties caused by lifestyle disturbances (e.g., sizes and timing of meals). Conversely, as the final value of meal boluses are normally decided by the patient, the meal bolus sizes calculated using the CR profile are used as references, thus a robust estimate of CR would be more practical from an application perspective. In this work, τ_{y} is selected to be 4 or the length of time elapsed before the next meal is taken if it is less than 4 hr, based on the observations of the UVA/Padova Simulator.

Parameter Optimization with Safety Constraints

To adapt the CR profile toward the target zone, a virtual optimization problem is formulated for each γₙ. The cost function is selected as the average postprandial BG level

yₙ^{γ}:=ƒₙ^{γ}(γₙ,θₙ^{γ}), (5)

Where ƒₙ^{γ}(γₙ, θₙ^{γ}) is used to represent the underlying unknown dependency of yₙ^{γ} on γₙ and other parameters θₙ^{γ}. The adaptation process is then performed by solving a sequence of constrained optimization problems with this unknown cost function:

min_{γ}_{ₙ}_{^{k+1}}ƒₙ^{γ}(γₙ^{k+1},θₙ^{γ}) (6)

s.t. |γₙ^{k+1}−γₙ^{k}|≤λ^{γ}γₙ^{k}, (7)

|ƒₙ^{γ}(γₙ^{k+1},θₙ^{γ})−ƒₙ^{γ}(γₙ^{k},θₙ^{γ})|≤δ^{y}^{ₙ}^{^{γ}}) (8)

γₙ^{k+1}≥γₙ^{k+1}. (9)

To ensure the smoothness of the adaptation procedure, three safety constraints are considered in the optimization problem above. The first and second constraints, respectively, restrict the rate of change of γₙ and ƒₙ^{γ}(γₙ^{k}, θₙ^{γ}). In this implementation, λ^{γ} is chosen as 30%, and δ^{y}^{ₙ}^{^{γ}} n is selected as 12 mg/dL. The third constraint directly bounds γₙ from below to avoid hypoglycemia risks caused by an underestimated CR; the lower bound γₙ^{k+1} is updated dynamically by taking the maximum value of γₙⁱ that has caused risk of hypoglycemia during the adaptation process. Note that γₙ^{k} and γₙ^{k+1} are known and ƒₙ^{γ}(γₙ^{k}, θₙ^{γ}) can be calculated based on historical CGM measurements when ƒₙ^{γ}(γₙ^{k+1}, θₙ^{γ}) is optimized for γₙ^{k+1} but the explicit expression of ƒₙ^{γ}(γₙ^{k+1}, θₙ^{γ}) is generally not known. To solve this problem, a data-driven BO-assisted algorithm will be provided below.

### Alternating BR and CR Adaptation

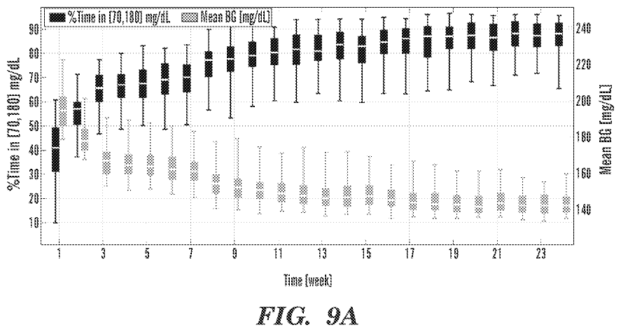

Considering the coupling effects of BR and CR on glucose management, the adaptation of {βₙ|n=1, 2, . . . , N} and {γₙ|n=1, 2, . . . , M} are performed in an interactive fashion. For safety consideration, only one profile (BR or CR) is adapted in each iteration. To do this, BR adaptation is performed in a time-driven manner such that the values of {βₙ|n=1, 2, . . . , N} are performed in a combined time- and event-triggered fashion; the adaptation procedure ends either when the zone objectives are achieved or when a maximum number of iterations N^{γ} is reached, which is set to 5 in this implementation. As meal boluses can be adjusted by the patients based on their knowledge and preference, the overall feedforward parameter adaptation phase begins by adapting the BR profile and iterates according to the alternating procedure described. An illustration of the procedure is provided in FIG. 8. The phase terminates when the zone objective criterion for CR adaptation remains valid after performing the BR adaptation.

### Choice of Parameters

A few parameters are adjustable in the proposed BR and CR adaptation procedure, including λ^{γ}, δ^{y}^{ₙ}^{γ}, N^{β} and N^{γ}, the values of which used in the in silico studies of this work have been provided in the above subsections. Specifically, λ^{γ} and δ^{y}^{ₙ}^{γ} are safety factors that limit the rates of change of the elements in the CR profile, while N^{β} and N^{γ} are the maximum allowable inner iterations to adapt BR and CR profiles (see also FIG. 8) and thus jointly determine the duration of the adaptation process. With these physical interpretations, other values of these parameters can be selected based on clinical experience and knowledge of particular patients in clinical studies.

### Learning Feedback Control Parameters

Phase II of the adaptation procedure deals with parameter adaptation for feedback control. As the appropriately adjusted BR and CR profiles in Phase I provide optimized operating conditions for the closed-loop controller, only moderate changes on the key parameters are needed to achieve satisfactory glucose regulation. Based on this observation, the approach here is to first determine the bottle-neck parameter that limits the performance of closed-loop control for a specific patient, and then to dynamically learn the appropriate value of the selected parameter. Ideally, improved performance could be potentially obtained by considering combined dynamic parameter selection and adaptation, but the improvement comes with compromised risk of hypoglycemia and time needed to complete the adaptation procedure.

Generally, parameter selection determines the parameter φ∈{\hat{R}, D̅, γ_{IOB}} to be adapted in Phase II. In this example, a sensitivity analysis approach is utilized to achieve automatic parameter selection, by rerunning the closed-loop control algorithm with different parameter settings using the most recent historical glucose measurements for a specific patient, which is the so-called advisory-mode analysis.²² To do this, for each φ∈{\hat{R}, D̅, γ_{IOB}} a pair of upper and lower bounds {φ⁺, φ⁻} that limit the range of feasible choices of φ in the adaptation procedure are specified; in our implementation, {φ⁺, φ⁻} is chosen as {150%, 50%}, {122%, 89%}, and {120%, 70%} of the corresponding nominal values for \hat{R}, D̅ and γ_{IOB} respectively. Advisory mode comparisons are performed by separately choosing φ=φ̅ and φ=φ and keeping parameters in {\hat{R},D̅,γ_{IOB}}/{φ} unchanged. The sensitivity analysis is then performed by solving

φ*=arg min_{φ∈{\hat{R},D̅,γ}_{_{IOB}}_{}}|I(φ⁺)−I(φ⁻)|, (10)

Where [symbol] (φ⁺) and [symbol] (φ⁻) denote the total amount of insulin calculated in the advisory mode analysis for φ=φ⁺ and φ=φ⁻, respectively. This procedure determines the parameter that has the “strongest” controllability of insulin delivery, hence the closed-loop glycemic regulation can be adjusted most efficiently.

### Parameter Optimization

The goal of adapting the selected parameter co is to achieve satisfactory average glucose level without having risk of hypoglycemia, which implies improved percentage time in euglycemic range [70, 180] mg/dl as the glucose profile is restricted below by considering constraints on hypoglycemia. To achieve this goal, we again formulate the adaptation procedure as a sequential optimization problem:

min_{φ}_{^{k+1}}ƒ^{φ}(φ^{k+1},θ^{φ}) (11)

s.t. |φ^{k+1}−φ^{k}|≤λ^{φ}φ^{k}, (12)

|ƒ^{φ}(φ^{k+1},θ^{φ})−ƒ^{φ}(φ^{k},θ^{φ})|≤δ^{y}^{^{φ}}, (13)

max{φ⁻,φ^{k+1}}≤φ^{k+1}≤min{φ⁺,φ̅^{k+1}}. (14)

Here, φ^{k+1} denotes the value of φ at the (k+1)th iteration, ƒ^{φ}(φ^{k+1}, θ^{φ}) denotes the average glucose value obtained using φ^{k+1} with θ^{φ} being potential dependent parameters. The constraints in Equations 12 and 13 restrict the rate of change of φ and ƒ^{φ}(φ,θ^{φ}), respectively. In our implementation, λ^{φ} is selected as 30%, and δ^{y}^{^{φ}} is chosen as 6 mg/dl; the roles of these two parameters are identical to those of λ^{γ} and δ^{y}^{ₙ}^{^{γ}} discussed in above. The constraints in Equation 14 bound the feasible region of φ; in addition to φ⁺, φ^{k+1} and φ̅^{k+1} provide additional dynamic bounds based on historical values of φ, namely, {φ¹, . . . , φ^{k}}, to help avoid hypoglycemia risks. The sequential optimization procedure ends either when average glucose level becomes satisfactory (less than 135 mg/dl in our implementation) or when the bounds in Equation 14 become active, the latter of which means the controller has achieved its performance limitation. Similar to the case of adapting γₙ, the analytical expression of the cost function ƒ^{φ}(φ^{k+1}, θ^{φ}) is not known. A BO-assisted optimization algorithm will be introduced to solve this problem.

### BO-Assisted Parameter Adaptation

Above, optimization problems with unknown objective functions and constraints need to be solved. Specifically, the two problems in Equations 6-9 and 11-14 share the form

min_{ψ}_{^{k}}ƒ(ψ^{k},θ^{ψ}) (15)

s.t., |ƒ(ψ^{k},θ^{ψ})−yₖ₋₁|≤δ, (16)

g(ψ^{k})≤0, (17)

where ƒ(ψ^{k}, θ^{ψ}) is an unknown function of ψ^{k} parameterized by θ^{ψ}, yₖ₋₁ is a noisy measurement of ƒ(ψ^{k-1}, θ^{ψ}), δ is a known parameter, and g(ψ^{k}) is a known (linear) function of ψ^{k}. Noticing the fact that a noisy measurement/estimate yₖ of ƒ(ψ^{k}, θ^{ψ}), which is average glucose level for a fixed ψ^{k}, is available, we solve this problem through designing a BO-assisted optimization algorithm, to make efficient use of historical measurements of {ƒ(ψ^{k}, θ^{ψ})|k∈πₖ} for an admissible set of iteration indexes πₖ; here, πₖ is defined as the set of iteration indexes for which the same set of parameters are adapted while other adaptation parameters are kept constant. The main idea is to obtain a data-driven estimate \hat{ƒ}(ψₖ, θₖ|Dₖ) of ƒ(ψ^{k}, θ^{ψ}) based on available historical data Dₖ. In the BO literature, different forms of \hat{ƒ}(ψₖ, θₖ|Dₖ) have been proposed.²³ As the size of data set for adaptation is small compared with those available for machine learning problems, a linear kernel is adopted in this work:

\hat{ƒ}(ψₖ,θₖ|Dₖ):=θ_{k,1}ψ^{k}+θ_{k,2} (18)

with θₖ:=[θ_{k,1}, θ_{k,2}]^{T}. Note that to make efficient use of data, the value of θₖ is made ψ-dependent and is obtained based on data segment D̅ₖ⊆Dₖ, which is the subset of data Dₖ corresponding to iteration indexes in πₖ. This further explains the rationale for using a linear kernel: the cost function in Equation 18 simply represents a local linearization of the unknown cost function ƒ(ψ^{k}, θ^{ψ}) around the adapted values of ψ^{k}.

To solve Equations 15-17, a BO-based algorithm is proposed (see Algorithm 1), which iteratively adapts tuning parameter ψₖ until the problem-dependent terminal conditions are satisfied. For each iteration, the algorithm starts with updating the admissible set πₖ, and obtains D̅ₖ⊆Dₖ based on πₖ (line 2 of the algorithm). For the problems in Equations 6-9 and 11-14, the effect of the adaptation parameter ψ on ƒ(ψ^{k}, θ^{ψ}) (which can be understood as the sign of the partial derivative) is known, and can be exploited to determine the search direction Sₖ (line 3); this helps ensure that the algorithm can evolve along the correct direction with noisy measurements {yₖ}.

If there is not enough data to perform data fitting (line 4), heuristic adjustment of φₖ will be performed along Sₖ for safety concerns, where δ^{S} is selected as 10% in our implementation. For all other scenarios, φₖ is adjusted through the proposed BO procedure (lines 5-11). The BO first estimates θₖ based D̅ₖ (line 6). With the obtained θₖ, ψₖ is calculated by solving a constrained optimization problem (line 7). As \hat{ƒ}(φₖ, θₖ|Dₖ) is linear with respect to φₖ, the optimization problem is actually a linear programming problem. To enhance the robustness of the algorithm against noises in {yₖ}, the deviation of ψₖ from ψₖ₋₁ is compared with Sₖ; the value of ψₖ will be recalculated if inconsistency is observed (line 8). Safety checks are further performed for ψₖ (line 10), where the value of ψₖ is truncated if the constraint is violated. The obtained φₖ is then implemented to obtain yₖ, which is collected to update Dₖ (line 12).

In this section, one can evaluate the proposed adaptation algorithm through multiple-month simulations on the 111-patient cohort of the US FDA accepted UVA/Padova simulator.¹⁶ To mimic intra-day variations in insulin sensitivity and the effect of dawn phenomenon, the diurnal patterns of the parameters in the endogenous glucose production and glucose utilization models are implemented according to Reference, 24 Inter-day variation in insulin sensitivity is further introduced by adding random noise obeying a uniform distribution within ±5% of the nominal values of the parameters that determine insulin sensitivity and dawn phenomenon.

To consider the effect of illness on insulin sensitivity, a healthy in silico patient has a 5% chance of entering a sick state that can last up to five consecutive days; when an illness event happens, it can either increase by 50% or decrease by 100% the magnitudes of the insulin sensitivity and dawn phenomenon parameters with probabilities of 0.5, throughout the illness period. One can assume that the state of sickness is not announced to the adaptation algorithm.

To mimic lifestyle disturbances, the in silico subjects take breakfast, lunch, and dinner with normally distributed meal sizes (with means and standard deviations equal to [50, 75, 75] g and [3, 4, 4] g carbohydrate [CHO]) and meal times uniformly distributed in [07:00, 09:00], [11:00, 13:00], and [18:00, 20:00], respectively; in addition, each meal can be skipped with probability 0.1. The CGM measurement noise is generated according to a random noise seed on each day.

One can assume that the meals are all fully announced but the meal boluses are calculated with potentially inappropriate CR profiles. The updated parameters obtained in each iteration are used for 1 week (7 days)¹⁰ before the next iteration, so that enough data can be collected for performance evaluation. For implementation purpose, one can assume the BR profile contains five segments, the effective period of which are [02:00, 05:00], [05:00, 10:00], [10:00, 16:00], [16:00, 21:00], and [21:00, 02:00], respectively; the CR profile are composed of four segments, the effective period of which are [05:00, 10:00], [10:00, 16:00], [16:00, 21:00], and [21:00, 05:00], respectively; only the first three segments that are responsible to breakfast (B), lunch (L), and dinner (D) are adapted and the segment that effective overnight is set to the default value from the simulator.

To evaluate the safety, effectiveness, and robustness of the adaptation algorithm, three scenarios are considered. In the first scenario (Scenario I), the patients are assumed to have doubled CR and halved BR profile segments compared with the default values in the simulator; both of these settings will lead to increased hyperglycemia due to conservative insulin delivery. In the second scenario (Scenario II), the patients are initiated with doubled CR and doubled BR profiles; the former would cause conservative meal boluses but the latter would counteract with relatively larger insulin microboluses, which makes it challenging for the adaptation algorithm to identify the appropriate tuning parameters. The third scenario (Scenario III) mimics real-life situations, in which different segments in the BR profile and CR profile

TABLE 1
Glycemic metrics obtained before and after the proposed adaptation algorithm

| | Scenario I | | | Scenario II | | | Scenario III | | |
|---|---|---|---|---|---|---|---|---|---|
| Metrics | Week 1 | Week 24 | p value | Week 1 | Week 24 | p value | Week 1 | Week 24 | p value |
| Percentage | 0.0 (0.0) | 0.0 (0.0) | .017* | 7.0 (7.6) | 0.0 (0.0) | <.001* | 0.0 (0.0) | 0.0 (0.0) | .763 |
| time < 54 | | | | | | | | | |
| mg/dL | | | | | | | | | |
| Percentage | 0.0 (0.0) | 0.0 (0.4) | <.001* | 15.7 (8.0) | 0.0 (0.1) | <.001* | 0.1 (1.0) | 0.0 (0.2) | .077 |
| time < 70 | | | | | | | | | |
| mg/dL | | | | | | | | | |
| Percentage | 41.1 (18.2) | 88.1 (9.5) | <.001* | 75.6 (9.9) | 88.8 (10.2) | <.001* | 81.6 (12.4) | 88.7 (13.8) | <.001* |
| time 70-180 | | | | | | | | | |
| mg/dL | | | | | | | | | |
| Mean BG | 194.3 (15.9) | 142.3 (8.8) | <.001* | 110.5 (10.8) | 142.0 (8.9) | <.001* | 144.3 (14.2) | 142.4 (9.1) | .756 |
| (mg/dL) | | | | | | | | | |

may deviate within a reasonable range from either above or below the corresponding appropriate values; to do this, the in silico subjects are initialized with randomized BR and CR profiles, in which the segments are generated according to uniform distributions within [50%, 150%] of the corresponding default values. For all in silico subjects, the zone MPC developed in Reference 20 with default parameters were used. The parameters in the adaptation algorithm are specified in Sections 2.1-2.3. For all three scenarios, the first week is utilized to collect data for initialization and thus no parameter is adjusted; the adaptation process starts from the second week. All simulations are run for 24 weeks. The key glycemic metrics obtained before and after the proposed adaptation algorithm are provided in Table 1.

### Results for Scenario I

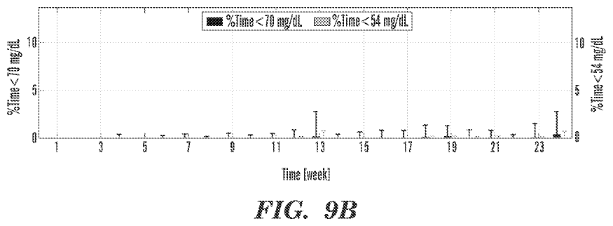

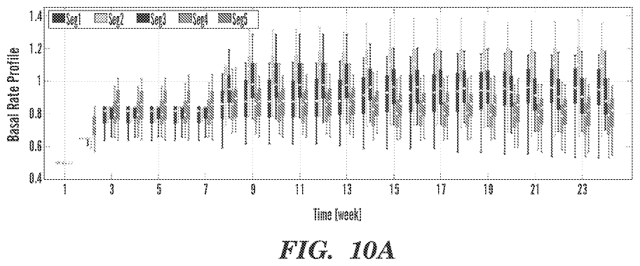

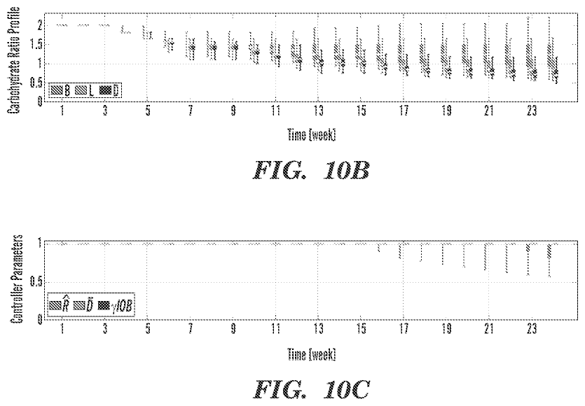

The results for Scenario I are shown in FIGS. 9A-9B and FIGS. 10A-10C. FIGS. 9A-9B provide the trends of key glycemic metrics during the adaptation procedure, including average BG levels, percentage time in the euglycemic range [70, 180] mg/dL, percentage time below 70 mg/dL, and percentage time below 54 mg/dL. Due to the joint effect of underestimated BR and overestimated CR profiles, the in silico subjects have elevated glucose levels on Week 1. From Week 2, monotonic and steady improvements are achieved by the proposed adaptation algorithm, and a trend of convergence in the performance metrics is observed around Week 12 as no significant changes happened in the rest of the simulations. For the 111-subject cohort, the adaptation algorithm manages to improve glycemic control performance dramatically in terms of both average glucose levels (from 194.3 mg/dL [Week 1] to 142.3 mg/dL [Week 24]; p<0.001) and average percent time in euglycemia range [70, 180] mg/dL (from 41.0% to 88.1%; p<0.001). The adaptation algorithm is safe in the sense that no hypoglycemia risk is caused during the process.

The trends of parameter changes during the adaptation procedure are provided in FIGS. 10A-10C. The important observation here is that through the use of statistical IOB constraints and smoothness constraints, strong performance is achieved by the BR profiles with their segments aligning around the reference value (which is 1 in FIGS. 10A-10C), instead of profiles with extremely large and small neighboring segments. In addition, the CR profile segments are aligned around the reference value; due to the safety constraints, risky (small) values for the CR segments are avoided. In addition, only moderate changes are observed for parameters in the feedback controller, which is expected as the obtained feedforward control parameters (BR and CR profiles) have set up an optimized operating point for the feedback controller, and thus the default values can achieve satisfactory glucose management. It is interesting to note that the second segment of the BR profile is larger than the other elements, which helps counteract against diurnal insulin sensitivity changes and dawn phenomenon.

### Results for Scenario II

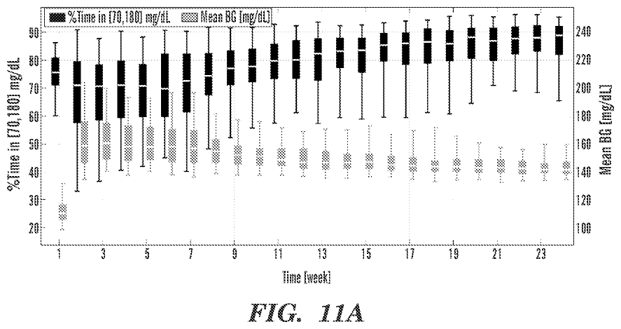

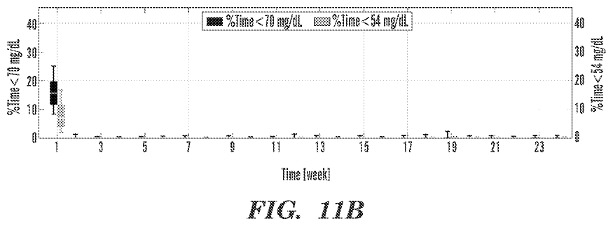

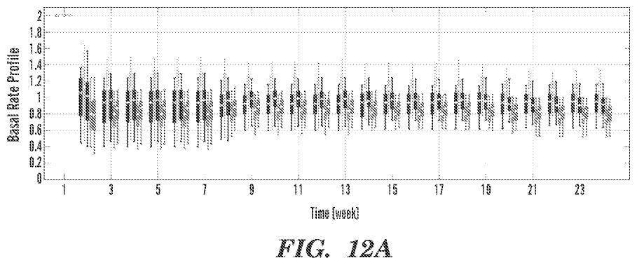

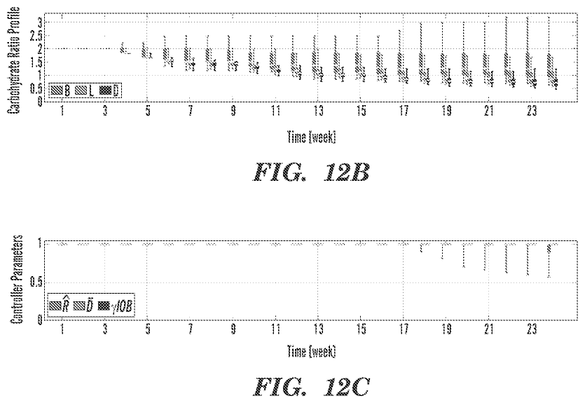

The results for Scenario II are provided in FIGS. 11A-11B and 12A-12C. Due to the increased BRs, severe hypoglycemia is observed on Week 1 of the simulation. The controller-led BR adaptation algorithm, however, is able to recognize safe BR profiles for the in silico cohort from Week 2 and perform safe fine tunes for the rest of the adaptation procedure. The CR adaptation procedure works properly as well, which manages to decrease the segments in the CR profiles for improved glycemic metrics. By eliminating risks of hypoglycemia (average percent time below 70 mg/dL, from 15.7% [Week 1] to 0.0% [Week 24], p<0.001), the adaptation algorithm improves average percent time in [70, 180] mg/dL (from 75.6% [Week 1] to 88.8% [Week 24], p<0.001). The obtained patterns of the BR profile and CR profile are similar to that of Scenario I.

### Results for Scenario III

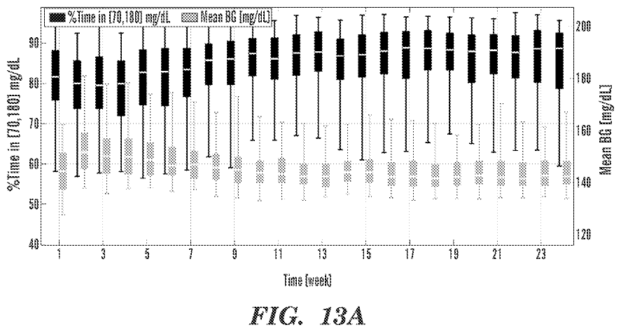

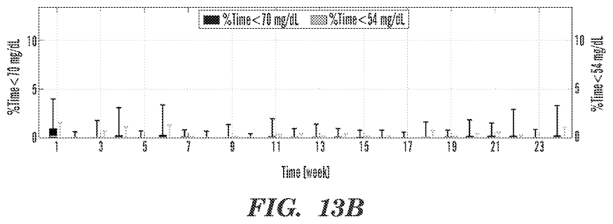

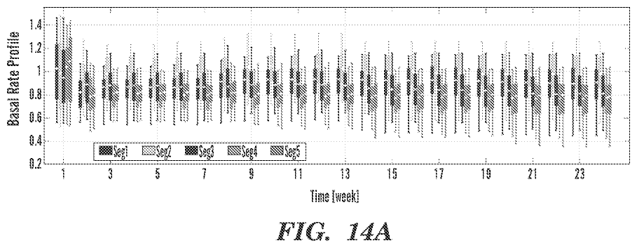

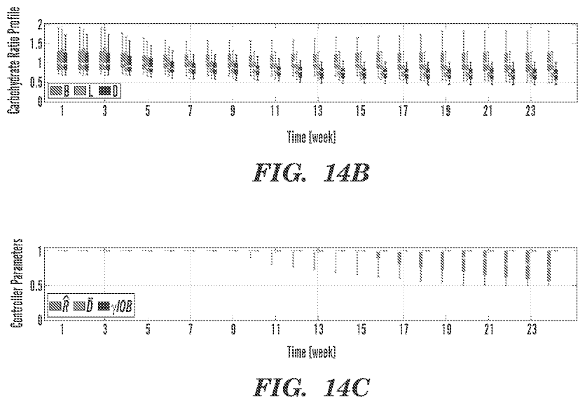

This scenario is devoted to simulate a 6-month study of AP parameter adaptation, for patients with mixed and reasonably over- or underestimated segments in their BR and CR profiles. The results are provided in FIGS. 13A-13B and 14A-14C. As expected, with slightly incorrect BR and CR profiles, the in silico patients generally have acceptable glucose management performance, which challenges the proposed adaptation method on the ability of recognizing and adjusting small but realistic mismatches in the adaptation parameters. From both figures, we observe that the algorithm is able to discern these parameter mismatches and achieve improved average percentage time in [70, 180] mg/dL (from 81.6% on Week 1 to 88.7% on Week 24), p<0.001, without causing risk of hypoglycemia. It appears that average percentage time below 70 mg/dL is decreased as well, but the significance is not sufficiently small (from 0.1% to 0.0%, p=0.077). Similar patterns in the BR and CR profiles to that obtained in the first two scenarios are realized.

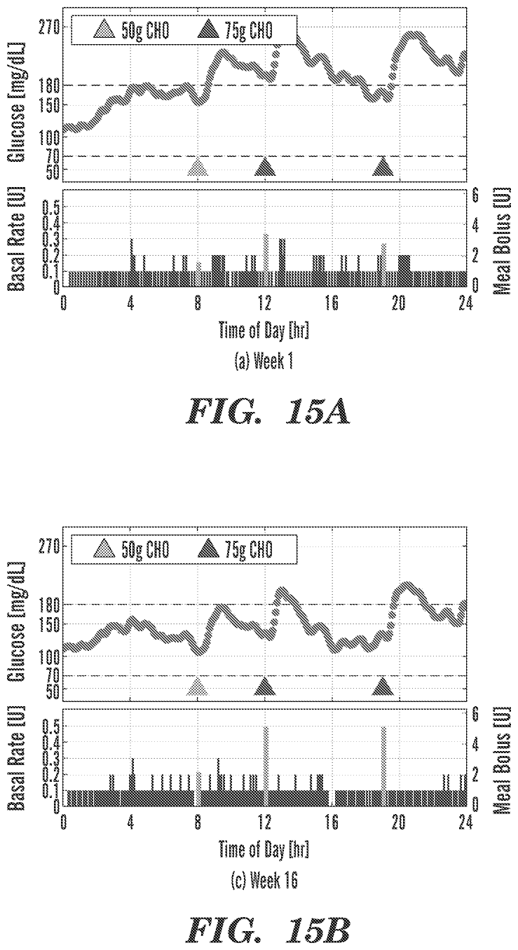

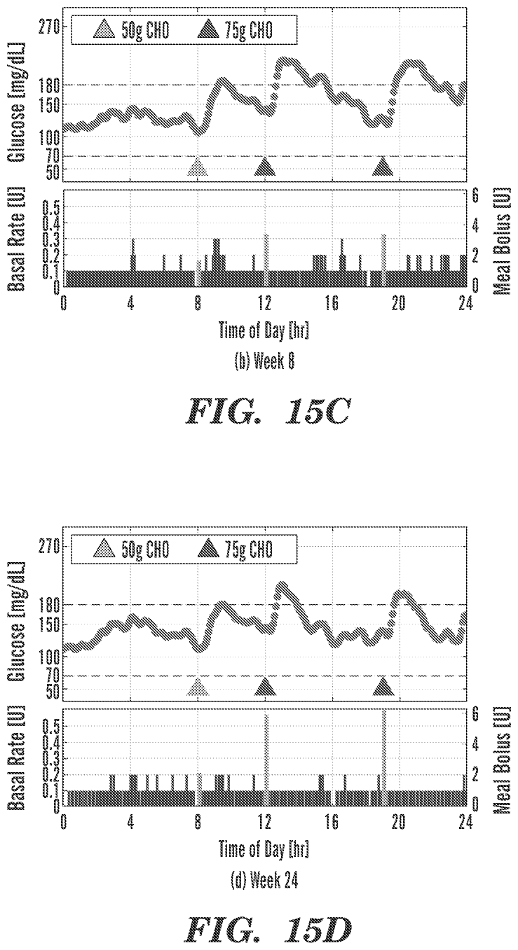

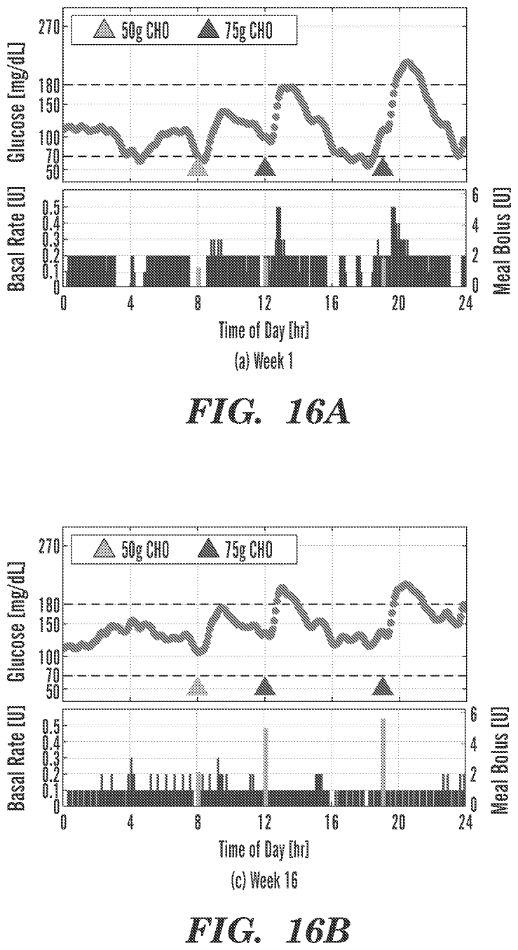

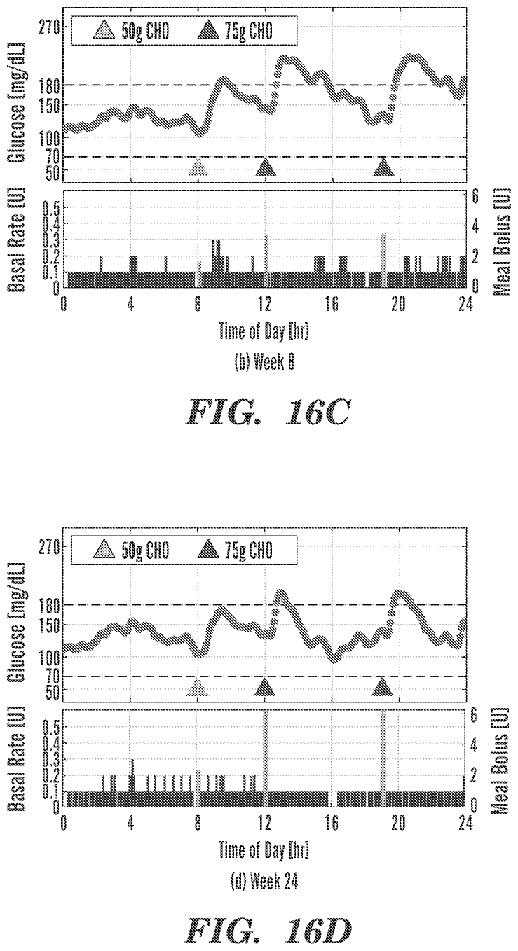

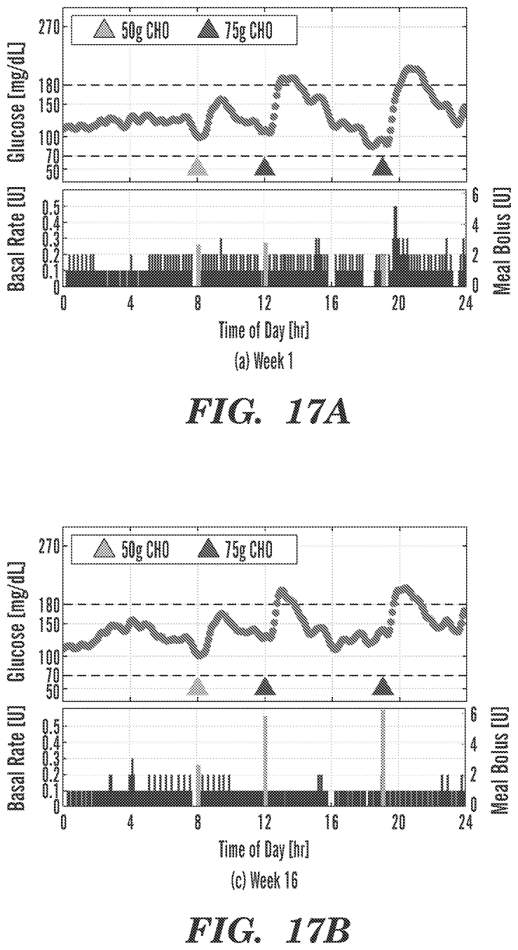

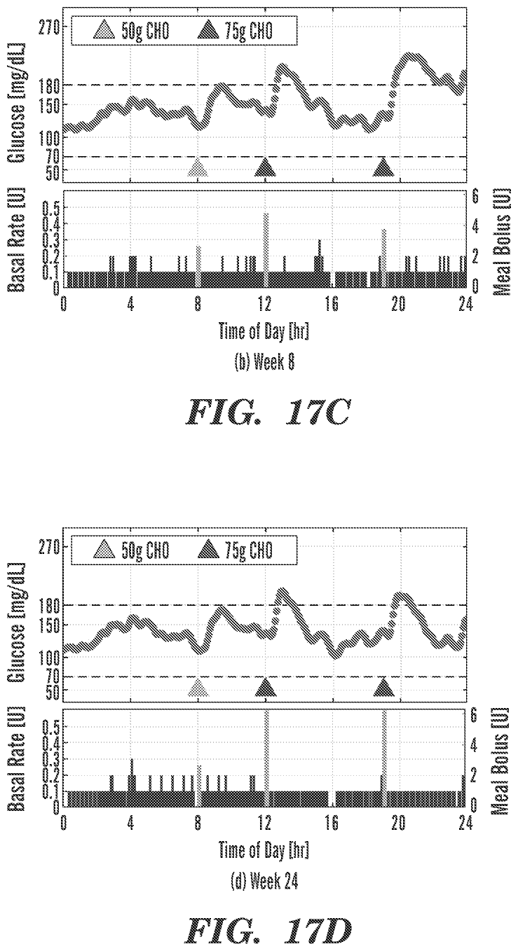

To further illustrate the effect of the adaptation process, glucose and insulin profiles of a particular patient simulated using the adaptation parameters obtained on Weeks 1, 8, 16, and 24 are provided in FIGS. 15A-15D (Scenario I), FIG. 16A-16D (Scenario II), and FIG. 17A-17D (Scenario III), in which a 24-hr protocol composed of a 50 g CHO breakfast at 08:00, a 75 g CHO lunch at 12:00, and a 75 g CHO dinner at 19:00 with the sensor noise seed is used. The results obtained are consistent with the population-level analysis in this section.

Several challenges exist in approaching the AP parameter adaptation problem. First, the effect of different AP parameters on glucose management is usually strongly coupled. For instance, the size of a real-time insulin microbolus is jointly determined by the BR profile and the closed-loop feedback controller, while BR, feedback controller, and user-requested meal boluses can all contribute to meal-related insulin delivery. This causes loss of identifiability to determine the correct root cause of poor glucose management; adjusting an incorrect parameter, however, may cause disastrous consequences. Second, lifestyle disturbances can also mask issues in glucose management. For instance, if a patient had a low BR for some time period while he/she happened to be jogging (which would increase insulin sensitivity) at the same time, the glucose traces might still appear to be acceptable, which would prevent an adaptation algorithm from identifying the real problems in glucose regulation. Finally, a considerable number of parameters in AP need to be considered as candidate variables in the adaptation procedure (which is 12 in this example's in silico tests in this work), while from a user experience perspective, the time of adaptation is limited (e.g., 24 weeks). As an iteration of the adaptation procedure may take 1 week to average out lifestyle disturbances, this basically implies that the task of AP adaptation through adjusting 12 coupled parameters need to be achieved within 24 iterations.

To overcome these challenges, a data-driven multivariate learning framework is developed in this example, on the basis of a dual-layer control scheme for long-term home use of AP. In this scheme, feed-back/feedforward control algorithms operate in the lower layer to achieve real-time glucose regulation, while the adaptation algorithm is implemented in the upper layer based on data from the lower layer.

The proposed adaptation procedure is composed of two phases. The first phase focuses on adaptation of feedforward control parameters, including BR and CR profiles. To bypass the non-identifiability issue, a controller-led approach is proposed for BR adaptation; the key idea is to exploit the intelligence from lower-layer feedback control algorithm to achieve autonomous decision of BR profiles. IOB and smoothness constraints are proposed to avoid hypoglycemia risks in the controller-led decision procedure. The CR profile is adapted through optimizing the postprandial BG levels toward a user-specified target zone while considering dynamically updated data-driven safety constraints. A hybrid time- and event-triggered iterating procedure is proposed to achieve joint adaptation of BR and CR profiles. The second phase is devoted to adapting feedback control behavior. To improve the efficiency of adaptation, a sensitivity analysis is proposed through performing advisory mode comparison based on historical glucose data,²² so that the bottleneck control parameter can be selected for adaptation. The selected parameter is then updated by optimizing average glucose levels while restricting risks of hypoglycemia.

For optimization problems encountered in CR profile and feedback control adaptation, analytical relationships between optimization parameters and performance metrics are generally not known, which precludes the use of standard optimization methods.²⁵ To overcome this additional challenge, the optimization problems are solved utilizing a BO approach, which was developed to optimize unknown objective functions based on noisy measurements in the machine learning community,²³ and has been recently used in on-line dynamics learning, automatic controller tuning and nonlinear adaptive control.26-29 In this example, BO is integrated with safety requirements and clinical experience, so that the “black-box” glucose regulation process can be safely adapted and improved.

The proposed adaptation method is evaluated on the basis of the 111-patient cohort of the U.S. FDA accepted UVA/Padova simulator¹⁶ for three in silico scenarios. The first two scenarios focus on edge cases with (a) underestimated BR and overestimated CR profiles and (b) overestimated BR and CR profiles, while the third scenario considers a real-life situation that different segments in the BR and CR profiles can be either overestimated or underestimated within a moderate extent. To challenge the proposed algorithm, lifestyle disturbances caused by timing and size changes for meals, inter- and intra-day insulin sensitivity changes, and occasionally occurring sickness that can significantly offset insulin sensitivity. This example demonstrates that for all scenarios, the proposed method is able to correctly identify and adaptively adjust the inappropriate parameters to achieve improved and satisfactory glucose regulation, without causing risks of hypoglycemia throughout the adaptation procedure.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### SELECTED EMBODIMENTS

Although the above description and the attached claims disclose a number of embodiments of the present invention, other alternative aspects of the invention are disclosed in the following further embodiments.

Embodiment 1. A system for managing the glucose of a patient, the system comprising: an artificial pancreas comprising a pump configured to deliver insulin into a patient; a memory containing machine readable medium comprising machine executable code having stored thereon; a glucose sensor configured to output glucose readings based on the blood glucose level of the patient; a memory containing machine readable medium comprising machine executable code having stored thereon; a control system coupled to the memory comprising one or more processors, the control system configured to execute the machine executable code to cause the control system to: periodically update a carbohydrate parameter of a control model based on a set of postprandial glucose readings previously output from the glucose sensor over a first time period; periodically update a basal rate parameter of the control model based on a set of fasting glucose readings previously output from the glucose sensor over a second time period; receive a set of glucose readings output from the glucose sensor; periodically process the set of glucose readings with the control model to determine an amount of insulin to deliver; and send a command to the artificial pancreas to deliver the amount of insulin using the pump.

Embodiment 2. Wherein the first and second time period is a week, two weeks, a few days, or five days.

Embodiment 3. Wherein the basal rate parameter and the carbohydrate parameter are sets of parameters or profiles.

Embodiment 4. Wherein updating the carbohydrate parameter comprises determining the carbohydrate parameter does not need to be updated.

Embodiment 5. Wherein updating the carbohydrate parameter further comprises first determining whether the carbohydrate parameter needs to be updated based on whether the set of postprandial glucose readings is outside a predefined threshold that indicates a change in a carbohydrate ratio of the patient.

Embodiment 6. Wherein updating the basal rate parameter further comprises first determine whether the basal rate parameter needs to be updated based on whether the set of fasting glucose readings is outside a predefined threshold that indicates a change in a basal rate of the patient.

Embodiment 7. Wherein the set of fasting glucose readings are output by the glucose sensor during a time of day while the patient is sleeping.

Embodiment 8. Wherein updating the basal rate parameter and the carbohydrate parameter is performed using a Bayesian optimization model.

Embodiment 9. Wherein the Bayesian optimization model comprises iterating changes to the basal rate parameter or the carbohydrate parameter until glucose values output by the glucose sensor are optimized over a third time period.

Embodiment 10. Wherein the Bayesian optimization model comprises a linear kernel.

Embodiment 11. Wherein the control model comprises a feedforward controller that calculates an amount of insulin to deliver based on a set of meal information provided by the patient.

Embodiment 12. Wherein the control model comprises a feedback controller that processes a set of real time glucose readings output from the glucose sensor to determine an amount of correction insulin to deliver.

Embodiment 13. A method of managing the glucose of a patient, the method comprising: updating a carbohydrate parameter of a control model based on a set of postprandial glucose readings previously output from the glucose sensor over a first time period; updating a basal rate parameter of the control model based on a set of fasting glucose readings previously output from the glucose sensor over a second time period; receiving a set of glucose readings output from the glucose sensor; processing the set of glucose readings with the control model to determine an amount of insulin to deliver; and sending a command to the artificial pancreas to deliver the amount of insulin using the pump.

Embodiment 14. Wherein updating a carbohydrate parameter and a basal rate parameter is performed weekly.

Embodiment 15. Wherein processing the set of glucose readings with the control model to determine an amount of insulin to deliver is performed at least two times a day.

Embodiment 16. Wherein processing the set of glucose readings with the control model to determine an amount of insulin to deliver is performed several times a day.

Embodiment 17. A non-transitory machine readable medium having stored thereon instructions for performing a method comprising machine executable code which when executed by at least one machine, causes the machine to: periodically update a carbohydrate parameter of a control model based on a set of postprandial glucose readings previously output from the glucose sensor over a first time period; periodically update a basal rate parameter of the control model based on a set of fasting glucose readings previously output from the glucose sensor over a second time period; receive a set of glucose readings output from the glucose sensor; periodically process the set of glucose readings with the control model to determine an amount of insulin to deliver; and send a command to the artificial pancreas to deliver the amount of insulin using the pump.

### CONCLUSION

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A system for managing the glucose of a patient, the system comprising:
an artificial pancreas comprising a pump configured to deliver insulin into a patient;
a memory containing machine readable medium comprising machine executable code having stored thereon;
a glucose sensor configured to output glucose readings based on the blood glucose level of the patient;
a memory containing machine readable medium comprising machine executable code having stored thereon;
a control system coupled to the memory comprising one or more processors, the control system configured to execute the machine executable code to cause the control system to:
periodically update a carbohydrate parameter of a control model based on a set of postprandial glucose readings previously output from the glucose sensor over a first time period;
periodically update a basal rate parameter of the control model based on a set of fasting glucose readings previously output from the glucose sensor over a second time period;
receive a set of glucose readings output from the glucose sensor;
periodically process the set of glucose readings with the control model to determine an amount of insulin to deliver; and
send a command to the artificial pancreas to deliver the amount of insulin using the pump.

2. The system of claim 1, wherein the first and second time period is a week, two weeks, five days, or 2 days.

3. The system of claim 1, wherein the basal rate parameter and the carbohydrate parameter are sets of parameters or profile.

4. The system of claim 1, wherein updating the carbohydrate parameter comprises determining the carbohydrate parameter does not need to be updated.

5. The system of claim 1, wherein updating the carbohydrate parameter further comprises first determining whether the carbohydrate parameter needs to be updated based on whether the set of postprandial glucose readings is outside a predefined threshold that indicates a change in a carbohydrate ratio of the patient.

6. The system of claim 1, wherein updating the basal rate parameter further comprises first determine whether the basal rate parameter needs to be updated based on whether the set of fasting glucose readings is outside a predefined threshold that indicates a change in a basal rate of the patient.

7. The system of claim 1, wherein the set of fasting glucose readings are output by the glucose sensor during a time of day while the patient is sleeping.

8. The system of claim 1, wherein updating the basal rate parameter and the carbohydrate parameter is performed using a Bayesian optimization model.

9. The system of claim 8, wherein the Bayesian optimization model comprises iterating changes to the basal rate parameter or the carbohydrate parameter until glucose values output by the glucose sensor are optimized over a third time period.

10. The system of claim 8, wherein the Bayesian optimization model comprises a linear kernel.

11. The system of claim 1, wherein the control model comprises a feedforward controller that calculates an amount of insulin to deliver based on a set of meal information provided by the patient.

12. The system of claim 1, wherein the control model comprises a feedback controller that processes a set of real time glucose readings output from the glucose sensor to determine an amount of correction insulin to deliver.

13. A method of managing the glucose of a patient, the method comprising:
updating a carbohydrate parameter of a control model based on a set of postprandial glucose readings previously output from the glucose sensor over a first time period;
updating a basal rate parameter of the control model based on a set of fasting glucose readings previously output from the glucose sensor over a second time period;
receiving a set of glucose readings output from the glucose sensor;
processing the set of glucose readings with the control model to determine an amount of insulin to deliver; and
sending a command to the artificial pancreas to deliver the amount of insulin using the pump.

14. The method of claim 15, wherein updating a carbohydrate parameter and a basal rate parameter is performed weekly.

15. The method of claim 15, wherein processing the set of glucose readings with the control model to determine an amount of insulin to deliver is performed at least two times a day.

16. The method of claim 15, wherein processing the set of glucose readings with the control model to determine an amount of insulin to deliver is performed several times a day.

17. A non-transitory machine readable medium having stored thereon instructions for performing a method comprising machine executable code which when executed by at least one machine, causes the machine to:
periodically update a carbohydrate parameter of a control model based on a set of postprandial glucose readings previously output from the glucose sensor over a first time period;
periodically update a basal rate parameter of the control model based on a set of fasting glucose readings previously output from the glucose sensor over a second time period;
receive a set of glucose readings output from the glucose sensor;
periodically process the set of glucose readings with the control model to determine an amount of insulin to deliver; and
send a command to the artificial pancreas to deliver the amount of insulin using the pump.
